(19) United States
(12) Patent Application Publication
(10) Pub. No.: US 2024/0189294 A1
(43) Pub. Date: Jun. 13, 2024

(54) METHOD FOR REGULATING SEBACEOUS GLAND

(71) Applicants: CHIN-LON LIN, Claremont, CA (US); BUDDHIST TZU CHI MEDICAL FOUNDATION, Hualien (TW)

(72) Inventor: Chung-Hsing CHANG, Hualien (TW)

(21) Appl. No.: 18/411,048

(22) Filed: Jan. 12, 2024

Related U.S. Application Data
(63) Continuation-in-part of application No. PCT/IB22/56437, filed on Jul. 12, 2022.
Application No. PCT/IB22/56437 is a continuation-in-part of application No. 17/372,649, filed on Jul. 12, 2021, now Pat. No. 11,752,151.

(51) Int. Cl. A61K 31/4439; A61K 31/404; A61K 31/506; A61P 17/08; A61P 17/14

(52) U.S. Cl. CPC A61K 31/4439; A61K 31/404; A61K 31/506; A61P 17/08; A61P 17/14

(57) ABSTRACT

A method for regulating sebaceous gland on an area of skin in a subject in need thereof is provided, which includes topically administering to the subject a casein kinase 1 inhibitor. The method can also treat the subject suffering from sebaceous gland-related disorder and/or hair loss. Accordingly, the present disclosure can bring about the effects on increasing hair growth and regulating sebaceous gland in the subject.

### CROSS REFERENCE TO RELATED APPLICATIONS

This application is a continuation-in-part of PCT Application No. PCT/IB2022/056437, filed on Jul. 12, 2022, which of U.S. application Ser. No. 17/372,649, filed on Jul. 12, 2021. The contents of these applications are incorporated herein by reference.

### BACKGROUND OF THE INVENTION

### 1. Field of the Invention

The present disclosure relates to methods for regulating sebaceous gland by inhibiting casein kinase 1 (CK1).

### 2. Description of the Prior Art

A sebaceous gland, comprised of sebocytes, is a small exocrine gland within the skin, which releases an oily or waxy substance known as sebum into a hair follicle. It has been known that sebaceous glands are androgen target tissues. In contrast to the pro-lipogenic actions of androgens, estrogens were originally described to have opposite effects to decrease the proliferation of sebocytes and to inhibit the production of sebum. The largest and most numerous sebaceous glands are found on the face, scalp, chest, and back. Sebum production is androgen dependent and begins at puberty in skin regions with abundant sebaceous follicles. This sebum serves to lubricate the skin and hair of mammals. The produced sebum aids in nourishing the hair shaft, preventing it from drying out, and minimizing friction between individual hairs. As a result, it contributes to the overall health and visual appeal of the hair.

Hair has important physiological functions. For example, hair coat keeps most mammals warm, dry and protected from harmful elements. Throughout the lifetime of an animal, hairs grow through a cyclic repetition of different phases of hair follicles, including telogen (a quiescence or resting phase), anagen (a regeneration or active hair growth phase) and catagen (a degeneration or transitional phase). At the end of telogen, the hair is shed, and a new hair replaces the original one, resulting in the start of the growing cycle again. Each follicle has its own life cycle that can be influenced by age, disease, and a wide variety of other factors.

However, acne, dermatitis, hair loss, or inadequate hair growth is a common problem experienced by many humans as well as many animals. The hair follicle cycle is regulated by both intrinsic and extrinsic signals which control quiescence and activation of hair follicle stem cells (HFSCs). Inadequate HFSC activation and proliferation underlie alopecia in numerous biological and pathological conditions, including aging. Molecules that can promote HFSC activation and anagen initiation have been intensely searched for, as they may both help reveal how sebaceous gland and hair regeneration are regulated and provide therapeutic and cosmetic interventions.

While there are approaches to this problem including such treatments as ultra-violet radiation, massage, psychiatric, drug and exercise therapy, none of these, however, has been generally accepted as being effective. Even approaches such as revascularization surgery and acupuncture have shown little, if any, promise.

Recently, many types of drugs ranging from vitamins to hormones have been tried, and only very few is recognized for successful therapeutic effects. For example, vitamin A and its derivatives (retinoids) have applied on treating acne or melasma. Nevertheless, research indicates that the application of retinoids is associated with symptoms such as dry skin, itching, peeling, cracking, cheilitis, as well as dryness in the eyes and nasal mucosa. In addition, as a precaution, vitamin A and its derivatives must not be used during pregnancy and by women planning to have a baby. With oral retinoids, there may be a possible risk of disorders such as teratism, depression, and anxiety. The frequently employed treatments available in the market, while inhibiting sebum production, concurrently result in hair loss. The most common approach for treating hair loss or sebum overproduction has been drug therapy. For instance, it has been known that androgenic hormone is used for the treatment for male pattern baldness; therefore, either systemic or topical application of an antiandrogenic hormone would provide an inhibiting action to prevent or treat the baldness. Nevertheless, antiandrogenic hormone is not as effective as expected.

As such, there remains an unmet need for providing compositions and methods for regulating sebaceous gland and/or enhancing hair growth in a subject in need thereof that are effective and easy for application.

### SUMMARY OF THE INVENTION

In the present disclosure, a method for regulating sebaceous gland on an area of skin in a subject in need thereof is provided, including topically administering to the subject a casein kinase 1 inhibitor.

In at least one embodiment, the subject suffers from a sebaceous gland-related disorder. In some embodiments, the subject suffers from a sebaceous gland-related disorder. In some embodiments, the sebaceous gland-related disorder is a sebaceous gland dysfunction and/or a sebum overproduction-related disorder. In some embodiments, the sebum overproduction-related disorder is induced by drug, infection, or hormones. In some embodiments, the sebum overproduction-related disorder is selected from the group consisting of an acne, a seborrhea, a dermatitis, and an oily skin. In some embodiments, the acne is a hormone-induced acne. In some embodiments, the dermatitis is an infection-induced dermatitis or a seborrheic dermatitis. In at least one embodiment, the regulating is reducing the size of the sebaceous gland. In at least one embodiment, the regulating is inhibiting adipogenesis and/or differentiation of sebocytes.

Also provided is a method for enhancing or stimulating hair growth on an area of skin in a subject in need thereof, including inhibiting casein kinase 1 in the area of skin. In at least one embodiment, the casein kinase 1 is inhibited during a telogen phase of a hair follicle cycle. In some embodiments, the casein kinase 1 is inhibited during an anagen phase of a hair follicle cycle. In some embodiments, the casein kinase 1 is inhibited in sebaceous gland. In some embodiments, the casein kinase 1 in the area of skin is casein kinase 1a. In at least one embodiment of the present disclosure, the subject suffers from hair loss. In some embodiments, the hair loss is a hair loss due to nutritional deficiency, a drug-induced hair loss, a radiation-induced hair loss, a stress-induced hair loss, a genetic hair loss, an aging hair loss or a disease-induced hair loss. In some embodiments, the drug-induced hair loss is induced by a chemotherapy drug, lithium, arsenic, bismuth, boric acid, thallium, colchicine, retinoid, heparin, warfarin, β-blocker, an angiotensin-converting enzyme (ACE) inhibitor, a hormone, valproic acid, carbamazepine, phenytoin, cimetidine, an antithyroid drug, a cholesterol-lowering drug, interferon, an anti-infective agent, amphetamine, an antidepressant, an anti-fungal agent, an anti-seizure agent, a birth control agent, a vitamin A-based medication, a medication for Parkinson's disease, a medication for stomach or a nonsteroidal anti-inflammatory drug. In some embodiments, the disease-induced hair loss is due to an autoimmune disease, a thyroid disorder, a metabolic syndrome, an infection or a cancer. In some embodiments, the autoimmune disease is alopecia areata, lupus erythematosus, Sicca syndrome, scleroderma, Crohn's disease, inflammatory bowel disease or psoriasis.

In some embodiments, the subject suffers from alopecia. In some embodiments, the alopecia is selected from the group consisting of androgenetic alopecia, alopecia areata, anagen effluvium, self-induced hair loss, telogen effluvium, and scarring alopecia.

In at least one embodiment of the present disclosure, inhibition of casein kinase 1 comprises inhibiting gene expression of casein kinase 1 in the area of skin. In at least one embodiment of the present disclosure, inhibition of casein kinase 1 comprises inhibiting gene expression of casein kinase 1α in the area of skin. In some embodiments, the inhibition comprises topical application of a casein kinase 1 inhibitor to the area of skin. In some embodiments, the casein kinase 1 inhibitor is selected from the group consisting of D4476, IC261, CKI7 and a compound represented by formulas II to VII below:

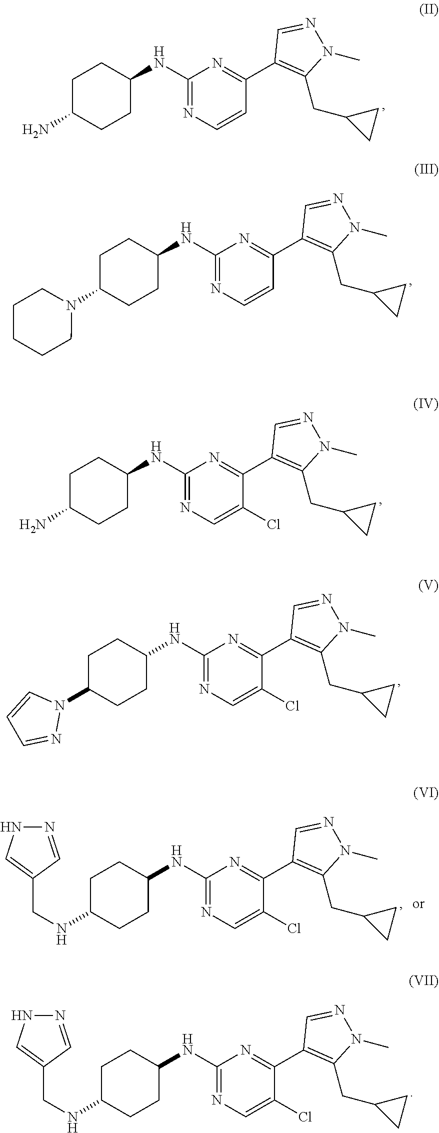

In some embodiments, hair loss is resulted by delayed or slow growth of hair, or premature falling of hair. In at least one embodiment, the present disclosure provides a method to treat hair loss in different stages of a hair follicle cycle. In at least one embodiment of the present application, a method for modulating a hair follicle cycle in a skin area of a subject in need thereof is provided, comprising inhibiting casein kinase 1 in the skin area. In some embodiments, the casein kinase 1 in the skin area is casein kinase 1a. In at least one embodiment of the present disclosure, the method for modulating a hair follicle cycle in a skin area of a subject in need thereof comprises inhibiting casein kinase 1α in the skin area. In an embodiment, the modulation of the hair follicle cycle is inducing the hair follicle cycle into an anagen phase, wherein the hair follicles are in a telogen phase before inhibition of casein kinase 1 in the skin area. In some embodiments, the modulation of the hair follicle cycle is prolonging an anagen phase of the hair follicles, wherein the hair follicles are in an anagen phase before inhibition of casein kinase 1 in the skin area.

The present disclosure also provides a method for increasing hair pigmentation in an area of skin in a subject in need thereof is provided, comprising inhibiting casein kinase 1 in the area of skin. In at least one embodiment of the present disclosure, the casein kinase 1 in the area of skin is casein kinase 1a. In at least one embodiment of the present disclosure, the area of skin comprises graying hair.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The present disclosure will become more readily appreciated by reference to the following descriptions in conjunction with the accompanying drawings.

FIG. 1A, FIG. 1B, FIG. 1C, and FIG. 1D show results of intraperitoneal (i.p.) tamoxifen (TMX) induction of CK1α ablation in keratinocytes of K14-Cre-ERT2-CK1α^{fl/fl} mice (CK1αKO) at 7 weeks old, the early phase of telogen. FIG. 1A shows the experiment scheme. FIG. 1B shows the phenotypes of mice at week 7 (W7) before and after shaving, 8 (W8), 9 (W9), 10 (W10), 11 (W11) and 12 (W12). FIG. 1C shows the H&E staining, and FIG. 1D shows the β-catenin staining of the skin samples harvested at indicated weeks of age.

FIG. 2A, FIG. 2B, FIG. 2C, FIG. 2D, and FIG. 2E show results of intraperitoneal (i.p.) tamoxifen (TMX) induction of CK1α ablation in keratinocytes of K14-Cre-ERT2-CK1α^{fl/fl} mice (CK1αKO) at 9 weeks old, the middle phase of telogen. FIG. 2A shows the experiment scheme. FIG. 2B shows the phenotypes of mice at week 9 (W9) before and after shaving, 10 (W10), 12 (W12), 13 (W13) and 16 (W16). FIG. 2C shows the H&E staining. FIG. 2D shows the β-catenin staining of the skin samples harvested. Arrows indicate the hair germs, where β-catenin was increased in the CK1αKO mice. FIG. 2E shows the BrdU staining.

FIG. 3A, FIG. 3B, FIG. 3C, and FIG. 3D show results of topical induction of CK1α ablation in keratinocytes of K14-Cre-ERT2-CK1α^{fl/fl} mice with 4-OH-TMX (CK1αKO). FIG. 3A shows the experiment scheme, with 4-OH-TMX topically applied to mice on the back of mice after shaving at week 9. Phenotypes were recorded, and samples were obtained at week 9 (W9), 11 (W11) and 13 (W13). FIG. 3B shows the phenotypes of mice at week 9 (W9), 11 (W11) and 13 (W13). FIG. 3C shows the H&E staining, and FIG. 3D shows the β-catenin staining of the skin samples harvested at indicated timing; “D” indicates number of days after induction.

FIG. 4A, FIG. 4B, FIG. 4C, FIG. 4D, FIG. 4E, and FIG. 4F show an exemplary CK1 inhibitor, A51, in inducing hair growth and pigmentation in C57/BL6 wild-type mice at 8 weeks old. FIG. 4A shows the experiment scheme, wherein A51 was topically applied on back skin of the mice for 2 or 3 times during week 8 after the back hair of mice were shaved. Arrows indicating at weeks 9, 10 and 11 show the timing for sampling. FIG. 4B shows the phenotypes of mice in weeks 8 (W8), 9 (W9), 10 (W10) and 12 (W12). 0.1*2 Q.O.D.: 0.1 mg each time every other day for a total of 0.2 mg; N indicates the number of mice in each group. FIG. 4C shows the H&E staining, and FIG. 4D shows the β-catenin staining of the skin samples harvested. FIG. 4E and FIG. 4F show Fontana-Masson staining of hair follicle pigmentation at high magnification and low magnification, respectively.

FIG. 5A, FIG. 5B, FIG. 5C, FIG. 5D, and FIG. 5E show CK1 inhibitors D4476 and IC261 in inducing hair growth and pigmentation in C57/BL6 wild-type mice at 8 weeks old. FIG. 5A shows the experiment scheme, in which D4476 or IC261 was topically applied on back skin of the mice every other day at 0.04 mg each time with a total of 0.12 mg applied. Arrows indicating at weeks 9 and 10 show the timing for sampling. FIG. 5B shows the phenotypes of mice in weeks 8 (W8), 9 (W9) and 10 (W10). FIG. 5C shows the H&E staining, and FIG. 5D shows the β-catenin staining of the skin samples harvested. FIG. 5E shows Fontana-Masson staining of hair follicle pigmentation.

FIG. 6A, FIG. 6B, and FIG. 6C show that inhibition of CK1 prolongs an anagen phase in a hair cycle. FIG. 6A shows the experiment scheme, with 4-OH-TMX topically applied to mice on the back of mice after shaving at week 4. FIG. 6B shows the phenotypes of mice in weeks 4 (W4), 6 (W6), 7 (W7) and 8 (W8), and FIG. 6C shows the result of H&E and β-catenin staining at week 8.

FIG. 7A, FIG. 7B, FIG. 7C, FIG. 7D, FIG. 7E, FIG. 7F, FIG. 7G, FIG. 7H, and FIG. 7I show that CK1a ablation in keratinocytes produces pigmented hair on Mc1r^{549del} mice. FIG. 7A shows the scheme of crossing to obtain the Mc1r^{549del} mice with CK1a ablation in keratinocytes. The Mc1r^{549del} mouse with yellow coat color was crossed with K14-CreER-CK1α^{f/f} mouse to generate K14-CreER-CK1α^{f/f};Mc1r^{549del} mouse. FIG. 7B shows the scheme of intraperitoneal injection of TMX to ablate CK1a in keratinocytes of the K14-CreER-CK1α^{f/f};Mc1r^{549del} mouse. FIG. 7C shows the phenotype of hair color on the back of Mc1r^{549del} and Mc1r^{549del} mice with CK1α ablation in keratinocytes (CK1α^{-/-};Mc1r^{549del}). FIG. 7D shows the hair shaft pigmentation under microscopy. FIG. 7E and FIG. 7F show the hair follicle pigmentation with Fontana-Masson staining and the number of Fontana-Masson-stained cells, respectively. FIG. 7G and FIG. 7H show the Western blotting analysis of expression levels of proteins in the KitL/c-Kit pathway in the back skin of mice and the quantitated expression levels in histograms, respectively. FIG. 7I shows eumelanin analysis on back skin of Mc1r^{549del} mouse versus CK1α^{-/-};Mc1r^{549del} mouse on day 14 and day 28.

FIG. 8A, FIG. 8B, FIG. 8C, and FIG. 8D show that effects of CK1 inhibition on sebaceous gland during enhancing hair growth. FIG. 8A shows intravital multiphoton imaging on the mouse ear of K14-CreER-CK1α; ROSA^{mT/mG}. FIG. 8B shows the WST-1 assay using SZ95 cell (human sebocyte cell line) treated with IC261 for 24 hr. FIG. 8C shows expressions of PPARγ (biomarker for adipogenesis) and c-Myc (biomarker for differentiation) in SZ95 cell treated with DMSO or IC261 at different concentrations. FIG. 8D shows IC261 does-dependently increased LC3B in treated SZ95 cells, indicating autophagy was induced.

### DETAILED DESCRIPTION

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the above examples for carrying out this disclosure without contravening its scope, for different aspects and applications.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein have to be defined based on the meaning of the terms together with the descriptions throughout the specification.

It is further noted that, as used in this disclosure, the singular forms “a,” “an,” and “the” include plural referents unless expressly and unequivocally limited to one referent. The term “or” is used interchangeably with the term “and/of” unless the context clearly indicates otherwise.

Also, when a part “includes” or “comprises” a component or a step, unless there is a particular description contrary thereto, the part can further include other components or other steps, not excluding the others.

The terms “subject,” “patient” and “individual” are used interchangeably herein and refer to a warm-blooded animal including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In some embodiments, the subject is a human.

The term “therapeutically effective amount” or “effective amount” is meant to include the amount of a compound that, when administered to a subject, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier” or “physiologically acceptable excipient” refers to a cosmetically or pharmaceutically acceptable material, composition, or vehicle, such as a liquid or solid filler, diluent, solvent, or encapsulating material. In some embodiments, each component is “pharmaceutically acceptable” in the sense of being compatible with the other ingredients of a cosmetic or pharmaceutical formulation, and suitable for use in contact with the tissue or organ of a subject (e.g., a human or an animal) without excessive toxicity, irritation, allergic response, immunogenicity, or other problems or complications, commensurate with a reasonable benefit/risk ratio. See, e.g., Remington: The Science and Practice of Pharmacy, 22nd ed.; Allen Ed.: Philadelphia, P A, 2012; Handbook of Pharmaceutical Excipients, 7th ed.; Rowe et al., Eds.; The Pharmaceutical Press and the American Pharmaceutical Association: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “about” or “approximately” means an acceptable error for a given value as determined by one of ordinary skill in the art, which depends in part on how the value is measured or determined. In some embodiments, the term “about” or “approximately” means within 1, 2, 3, or 4 standard deviations. In some embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “drug,” “cosmetic agent” and “therapeutic agent” refer to a compound or a cosmetical or pharmaceutical composition thereof, which is administered to a subject for preventing, ameliorating or treating one or more symptoms of a disorder, disease, or condition.

In some embodiments of the present disclosure, the methods provided herein comprise treating a subject regardless of patient's age, although some diseases or disorders are more common in certain age groups.

CK1 inhibitors (CKIs) of the present disclosure may have at least twice, at least 5 times, or at least 10 times the inhibitory activity towards CK1 as compared to other kinases such as cyclin-dependent kinases (CDKs) regulating the cell cycle, (e.g., Cdk2, Cdk4, and Cdk6). In addition, CK1 inhibitors have at least twice, at least 5 times, or at least 10 times the inhibitory activity towards CK1 as compared to protein kinase C (PKC), protein kinase A (PKA), epidermal growth factor receptor 2 (HER2), rapidly accelerated fibrosarcoma 1 (RAF-1), mitogen-activated protein kinase 1 (MEK1), mitogen-activated protein kinase (MAP kinase), epidermal growth factor receptor (EGF receptor), platelet-derived growth factor receptor (PDGF receptor), insulin-like growth factor receptor (IGF receptor), phosphoinositide 3 kinase (PI3 kinase), Wee1 kinase, Src, and/or Abl.

In some embodiments of the present disclosure, CKIs are selective towards CK1α (CSNK1A; at the genomic, mRNA or protein level, GenBank Accession Nos. NP_001020276, NM_001025105 and NM_001020276). In some embodiments of the present disclosure, CKIs also have inhibitory activity towards other CK isozymes such as CK1δ, CK1ε, etc. In at least one embodiment of the present disclosure, a CKI has inhibitory activity towards CK1α, CK1δ, CK1ε, and any combination thereof.

In at least one embodiment of the present disclosure, a casein kinase 1 inhibitor is represented by the following general formula I, including any stereoisomer or salt thereof:

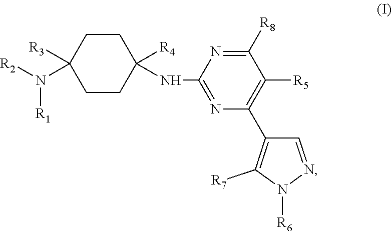

wherein:
R₁ and R₂ are each independently selected from the group consisting of H, straight or branched C1-C8 alkyl, straight or branched C1-C5 alkoxy, straight or branched C1-C5 acyl, C5-C15 aryl, and C3-C7 heteroaryl each optionally substituted by at least one of halide, hydroxyl, ester, ether, C5-C15 aryl, C3-C7 heteroaryl, and amide; or R₁ and R₂ together with the nitrogen atom they are connected to form a 4 to 7 membered saturated, unsaturated or aromatic ring that may optionally include at least one of N, O, NH, C═N, C═O and SO₂ and can optionally be substituted with at least one of straight or branched C1-C5 alkyl, C5-C15 aryl, C3-C7 heteroaryl, hydroxyl, halide and cyano;
R₃ and R₄ are each independently selected from the group consisting of H, straight or branched C1-C8 alkyl optionally substituted by at least one of halide, hydroxyl, alkoxy, C5-C15 aryl, C3-C7 heteroaryl, ester and amide; or
R₁ or R₂ together with R₃ and the carbon and nitrogen atom they are each connected to form a 4 to 7 membered saturated, unsaturated or aromatic ring that may optionally include at least one of N, NH, 0, C═N, C═O, and SO₂, and can optionally be substituted with at least one of straight or branched C1-C5 alkyl, C5-C15 aryl, C3-C7 heteroaryl, hydroxyl, carbonyl, and halide;
R₅ and R₈ are each independently selected from the group consisting of H, halide, straight or branched C1-C8 alkyl, straight or branched C2-C8 alkenyl, and straight or branched C2-C8 alkynyl optionally substituted by at least one halide;
R₆ is selected from straight or branched C1-C8 alkyl, straight or branched C2-C8 alkenyl, straight or branched C2-C8 alkynyl, C5-C10 cycloalkyl, and saturated or unsaturated 4 to 6 membered heterocycle optionally substituted by at least one of straight or branched C1-C8 alkyl, C3-C7 cycloalkyl, 4 to 6 membered heterocycle, C5-C15 aryl, C3-C7 heteroaryl, halide, hydroxyl, and C1-C5 alkyl halide;
R₇ is selected from the group consisting of straight or branched C1-C8 alkyl, straight or branched C2-C8 alkenyl, and straight or branched C2-C8 alkynyl optionally substituted by at least one of C3-C7 cycloalkyl, 4 to 6 membered heterocycle, C5-C15 aryl, C3-C7 heteroaryl, halide, hydroxyl, and C1-C5 alkyl halide.

Additional casein kinase I inhibitors include those described in International Publication No. WO 2017/021969; the disclosure of which is incorporated herein by reference in its entirety.

The cosmetical or pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the cosmetical or pharmaceutical compositions provided herein can also comprise liposomes, micelles, microspheres, nanosystems, and any mixtures thereof.

Cosmetically or pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The cosmetical or pharmaceutical compositions can also be administered topically by electroporation, iontophoresis, phonophoresis, sonophoresis, microneedle or needle-free injection, such as PowderTect (Chiron Corp., Emeryville, CA) and Bioject (Bioject Medical Technologies Inc., Tualatin, OR).

The cosmetical or pharmaceutical compositions provided herein can be provided in the forms of ointments, creams, and gels. Suitable ointment vehicles include oleaginous or hydrocarbon vehicles, including lard, benzoinated lard, white petrolatum, olive oil, cottonseed oil, and other oils; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, e.g., Remington: The Science and Practice of Pharmacy). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Suitable gels may be semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and Carbopol; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gums; sodium alginate; and gelatin. In order to prepare a uniform gel, dispersing agents such as alcohol or glycerin can be added, or the gelling agent can be dispersed by trituration, mechanical mixing, and/or stirring.

### Example

Exemplary embodiments of the present disclosure are further described in the following examples, which should not be construed to limit the scope of the present disclosure.

Generally, the nomenclature used herein and the laboratory procedures utilized in the present disclosure include molecular, biochemical and recombinant DNA techniques. Such techniques are thoroughly explained in the literature. See, for example, “Molecular Cloning: A Laboratory Manual,” Sambrook et al., (1989); “Current Protocols in Molecular Biology,” Volumes I-III Ausubel, R. M., ed. (1994); “A Practical Guide to Molecular Cloning,” John Wiley & Sons, New York (1988); Watson et al., “Recombinant DNA,” Scientific American Books, New York; Birren et al. (eds) “Cell Biology: A Laboratory Handbook,” Volumes I-III Cellis, J. E., ed. (1994); “Culture of Animal Cells A Manual of Basic Technique,” Freshney, Wiley-Liss, N.Y. (1994), Third Ed.; “Transcription and Translation,” Hames, B. D., and Higgins S. J., eds. (1984); “Animal Cell Culture,” Freshney, R. I., ed. (1986); “Immobilized Cells and Enzymes,” IRL Press, (1986); “A Practical Guide to Molecular Cloning,” Perbal, B., (1984) and “Methods in Enzymology,” Vol. 1-317, Academic Press; “PCR Protocols: A Guide to Methods and Applications,” Academic Press, San Diego, Calif (1990); Marshak et al., “Strategies for Protein Purification and Characterization A Laboratory Course Manual,” CSHL Press (1996); all of which are incorporated by reference as if fully set forth herein. Other general references are provided throughout this disclosure. The procedures therein are believed to be well known in the art and are provided for the convenience of the reader. All the information contained therein is incorporated herein by reference.

### Materials and Methods

### Mice

K14-Cr-ERT2-CK1α^{fl/fl} mice, Mc1r^{549del} mice and Mc1r^{549del} mice with CK1α ablation in keratinocytes (CK1α^{-/-}Mc1r^{549del}) were collaboratively generated in National Laboratory Animal Center (NLAC) in Tainan, Taiwan, and wild type C57BL/6J mice were also purchased from NLAC. Experiments and animal caring were performed in the Laboratory Animal Center, Tzu Chi University, Hualien, Taiwan, with the approval of Animal Care Committee in accordance with legal and ethical standards. Both male and female mice were used. The hair cycle in wild C57BL/6J mice is related to the age; weeks 4 to 6 are an anagen phase, weeks 6 to 7 are a catagen phase, and weeks 7 to 12 are a telogen phase.

### Targeting Gene Activation (Induction of CK1α Ablation)

For deletion of CK1α expression in keratinocytes, K14-Cre-ERT2-CK1α^{fl/fl} mice were treated with 100 mg/kg tamoxifen (TMX) dissolved in corn oil (C8267; Sigma) two times by intraperitoneal injection, or topically treated with 4-hydroxyl-tamoxifen (4-OH-TMX) (H6278; Sigma), which was dissolved in 99.9% alcohol (32205; Sigma). 200 μL with the concentration of 3 mg/mL was administered to the back skin after shaving for 5 times/week.

### Preparation of CK1 Inhibitors (CKI)

A51 is a gift from Dr. Yinon Ben-Nerian's lab (which was published in Cell, Volume 175, Issue 1, 20 Sep. 2018, pages 171-185). For topical application onto mouse skin, 1 mg of CKI was dissolved in 6 μL dimethyl sulfoxide (DMSO) and mixed with 54 μL of vehicle which contained 60% of white wax and 40% of paraffin oil. The final working concentration is 0.1 mg/cm².

D4476 was purchased from Sigma Aldrich (#D1944). For topical application onto mouse skin, 4 mg of CKI was dissolved in 1 mL DMSO as the stock. 10 μL stock was mixed with 40 μL of vehicle which contained 60% of white wax and 40% of paraffin oil. The final working concentration is 0.04 mg/cm².

IC261 was purchased from Sigma Aldrich (#40090). For topical application onto mouse skin, 4 mg of CKI was dissolved in 1 mL DMSO as the stock. 10 μL stock was mixed with 40 μL of vehicle which contained 60% of white wax and 40% of paraffin oil. The final working concentration is 0.04 mg/cm².

### Topical Drug Treatment

Mice at 8 weeks old were shaved to remove hair on back skin and cleaned with 75% ethanol. Topical CKIs were applied on the back skin 3 times in one week, and the tissue was harvested on day 7, day 14 and some on day 28 after application of CKIs. The tissue was analyzed by the methods described below.

### Histopathology, Immunohistochemical Staining, and Fontana-Masson Staining

Skin samples were harvested at indicated timings for tissue analysis. Paraffin-embedded specimens were cut into 5-μm sections. After deparaffinization (with tissue slides being soaked in xylene three times, each time for 5 min) and rehydration (with slides being incubated in the following graded series of ethanol: 100%, 100%, 95%, 90% and 70%, 5 min each), the slides were rinsed with distilled water for 5 min.

### Hematoxylin & Eosin (H&E) Staining and Fontana-Masson Staining

Fontana-Masson staining and H&E staining were performed by using the kit from ScyTek Laboratories, Inc. according to the manufacturer's instructions.

Briefly, for Fontana-Masson staining, a freshly mixed ammoniacal silver solution was placed in a 58° C. to 60° C. water bath and allowed to equilibrate. The slides were incubated in a warmed ammoniacal silver solution for 30 to 60 min or until the tissue sections became yellow/brown in color, and were then rinsed with distilled water for 3 to 5 seconds (s). The slides were incubated in a gold chloride solution (0.2%) for 30 s and were then rinsed with distilled water for 3 to 5 s. The slides were incubated in a sodium thiosulfate solution (5%) for 1 min and were then rinsed with distilled water for 3 to 5 s. The slides were incubated in a nuclear fast red solution for 5 min, rinsed for 1 min in running tap water, dehydrated in four changes of fresh absolute alcohol (95%, 95%, 100% and 100%) and xylene, and mounted with Histokitt (Assistent).

For histopathology by H&E staining, skin tissues were fixed overnight in 10% neutral buffered formalin at 4° C. and then transferred to 70% ethanol before being processed and embedded in paraffin. Paraffin sections were then stained with H&E.

### Immunohistochemical Staining

Paraffin sections were incubated in a humidity chamber for 15 min at 60° C. Sections were deparaffinized in two changes of xylene for 5 min each and hydrated in two changes of 100% ethanol for 5 min each, then in 95% and 80% ethanol for 5 min each, and finally rinsed in distilled water. Antigen retrieval was enhanced by microwaving the slides in a citrate buffer (DAKO; pH 6.0) for 20 min. Endogenous peroxidase activity was quenched with 3% hydrogen peroxide in methanol. After blocking, sections were incubated overnight at 4° C. with β-catenin antibody (1/100; BD Bioscience). Secondary antibodies used were horseradish peroxidase (HRP)-polymer anti-mouse antibodies (Nichirei). 3-Amino-9-ethylcarbazole (AEC) chromogen (ScyTek) was used for detection, and hematoxylin was used as a counterstain.

5-bromo-2′-deoxyuridine (BrdU) Staining

For labelling of proliferating cells in a mouse, intraperitoneal injection of 200 μL BrdU (Amersham; cell proliferation labelling reagent, RPN201, GE Healthcare) was performed. Six hours later, skin samples were harvested for paraffin embedding. 4 m paraffin embedded sections were prepared as above, and then incubated with Target Retrieval Solution (pH 6.0) (DAKO, S1699) in a water bath for 20 min at 95° C. to 120° C. The staining dish was moved to room temperature, allowing the slides to cool to 30° C., followed by rinsing with PBS once. Then, the slides were incubated with primary antibody BrdU (Thermo Fisher Scientific, B23151, 1:100) at 4° C. overnight. The slides were rinsed with PBS for 3 times, 5 min each time. The slides were incubated with EnVision/HRP, Rabbit/Mouse (ENV) (DAKO, k5007) for 30 min, and rinsed with PBS for 3 times, 5 min each time. Then, the slides were incubated with AEC+ High Sensitivity Substrate Chromogen Ready-to-Use (DAKO, K3461) for 5 min, and washed with running water for 5 min. Then, the slides were counterstained with hematoxylin for 30 s to 1 min, and washed with running water for 10 min. The slides were mounted with Aqueous-Mount (ScyTek, 51934).

### Western Blot

For experiments involving tissues, the tissues were homogenized and lysed in 1× tissue protein extraction reagent (Thermo Fisher) containing protease inhibitor (Millipore) and kept on ice; for experiments involving cells, cell pellet was collected and lysed in 1×radioimmunoprecipitation assay (RIPA) lysis buffer containing protease inhibitor (Millipore) and kept on ice. The total protein concentration was determined using Protein Assay Dye Reagent Concentrate (Bio-Rad) following manufacturer's instruction with a microplate reader. Sample was diluted in SDS-PAGE Sample Buffer (Bio-Rad) and heated at 95° C. for 5 minutes. The protein sample was then resolved on SDS-PAGE gels (TGX FastCast acrylamide solutions, Bio-Rad) and transferred onto poly(vinylidene fluoride) (PVDF) membranes (Millipore). 5% BSA was used, and then the membranes were incubated with primary antibodies overnight at 4° C. The membranes were then washed by Tris-buffered saline with Tween 20 (TBST) and incubated with secondary antibodies for 1 hour at room temperature. The signal was detected using iBright FL1000 Imaging System from Thermo Fisher Scientific.

### Antibodies

The antibodies used in immunohistochemistry (IHC) and Western blotting (WB) in this disclosure were listed in Table 1 below, where manufacturers, catalogue number and the dilution ratio used were provided.

TABLE 1

| Antibody | Manufacturer | Number | WB | IHC |
|---|---|---|---|---|
| KitL | Abcam, UK | Ab64677 | 1:2,000 | |
| Rabbit anti-c-Kit | GeneTex | GTX33947 | 1:1,000 | 1:200 |
| (CD117) | | | | |
| MiTF (SPM290) | Santa Cruz | sc-56433 | 1:1,000 | 1:50 |
| | Biotechnology, | | | |
| | Inc., USA | | | |
| Purified mouse | BD, USA | 61054 | 1:1,000 | 1:200 |
| anti-β-catenin | | | | |
| Tyrosinase (T311) | Santa Cruz | sc-20035 | 1:1,000 | |
| | Biotechnology, | | | |
| | Inc., USA | | | |
| p53(1C12) mouse | Cell Signaling | #2524 | 1:1,000 | |
| mAb | Technology, USA | | | |
| Purified mouse | BD, USA | 61054 | 1:1,000 | 1:200 |
| anti-β-catenin | | | | |
| Glyceraldehyde 3 | Cell Signaling | #2118 | 1:2,000 or | |
| phosphate | Technology, USA | | 1:10000 | |
| dehydrogenase | | | | |
| (GAPDH) | | | | |
| PPARγ | Santa Cruz | Sc-7273 | 1:1000 | |
| | Biotechnology, | | | |
| | Inc., USA | | | |
| c-Myc | Cell Signaling | #9402 | 1:1000 | |
| | Technology, USA | | | |
| LC3B | Cell Signaling | #3368 | 1:1000 | |
| | Technology, USA | | | |
| Donkey IgG-heavy | Bethyl | A140-107D4 | | 1:500 |
| and light chain | Laboratories | | | |
| antibody | Inc., USA | | | |
| Donkey IgG-heavy | Bethyl | A140-107D2 | | 1:500 |
| and light chain | Laboratories | | | |
| antibody | Inc., USA | | | |
| Goat anti-rabbit | Millipore, | AP132P | 1:5,000 | |
| IgG, peroxidase | Germany | | | |
| conjugated | | | | |
| Goat anti-mouse | Millipore, | AP124P | 1:5,000 | |
| IgG, peroxidase | Germany | | | |
| conjugated | | | | |

### Eumelanin Analysis

Skin samples were measured and rinsed with Ca²⁺-, Mg²⁺-free PBS (pH 7.4) to remove blood. For assays of melanin content, the tissues were minced with scissors and homogenized in 10 volumes of PBS at 28° C. Samples of skin were processed for chemical analyses of eumelanin to detect the specific degradation product, pyrrole-2,3,5-tricarboxylic acid (PTCA). One nanogram of PTCA corresponds to 50 ng of eumelanin. The statistical significance of differences in the contents of eumelanin was determined by Student's t test for comparisons of groups of equal size.

### Establish K14-CreER-CK1a; ROSAmT/mG Mouse

The K14-CreER-CK1a; ROSAmT/mG mouse was generated by crossing K14-CreER-CK1α and mT/mG mice in National Laboratory Animal Center in Tainan and kept under specific pathogen free conditions. For CK1α ablated-keratinocyte labeling, we performed intraperitoneal injection of TMX which was dissolved in corn oil with dose of 1 mg/day for continuous 5 days. The red fluorescence (tdTomato; mT) on keratinocyte would be turned to green fluorescence (EGFP; mG) when CK11a was ablated through TMX induction.

### Intravital Multiphoton Imaging of the Mouse Ear Skin

We performed intravital multiphoton imaging on the mouse ear at Dr. Peilin Chen's Lab (Research Center for Applied Sciences, Academia Sinica, Taipei, Taiwan). The method and procedure was modified according to doi: 10.1038/nprot.2011.438. The peak emission wavelength of EGFR (green fluorescence) and tdTomato (ROSA26) (red fluorescence) is 510 and 580 nm, respectively.

### SZ95 Cell Culture and WST-1 Assay

SZ95 cells (human sebocyte cell line) were maintained in Sebomed basal Medium (Sigma) supplemented with 10% FBS and 5 ng/mL epidermal growth factor under standard cell culture conditions of 37° C. and 5% CO₂.

WST-1 assay. Seeding cells with density 1.5×10⁴ cells/well in 96-well plate, incubate overnight. Remove medium and add 100 μL fresh medium contain various amount of IC261 (1, 50, 100, 200, 500, 1000 nM) in each well, incubate for 24 hr. Add 10 μL/well WST-1, incubate for 4 hr. Measure the absorbance of OD 450 nm and OD 655 nm by ELISA reader.

### Statistical Analysis

The statistical analysis in this disclosure present analysis result as mean±standard deviation. Student t test was applied for comparison between groups. P-values less than 0.05 were considered statistically significant.

### Example 1: Inhibition of CK1α Induces an Anagen Phase in a Hair Cycle

CK1α inhibitions in mice keratinocytes were carried out by various means, and all shown to induce anagen in a hair cycle from a telogen phase, early telogen (7 weeks) and middle telogen (9 weeks).

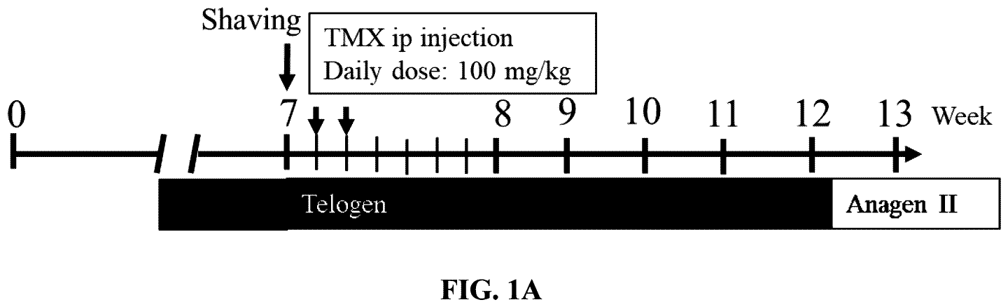

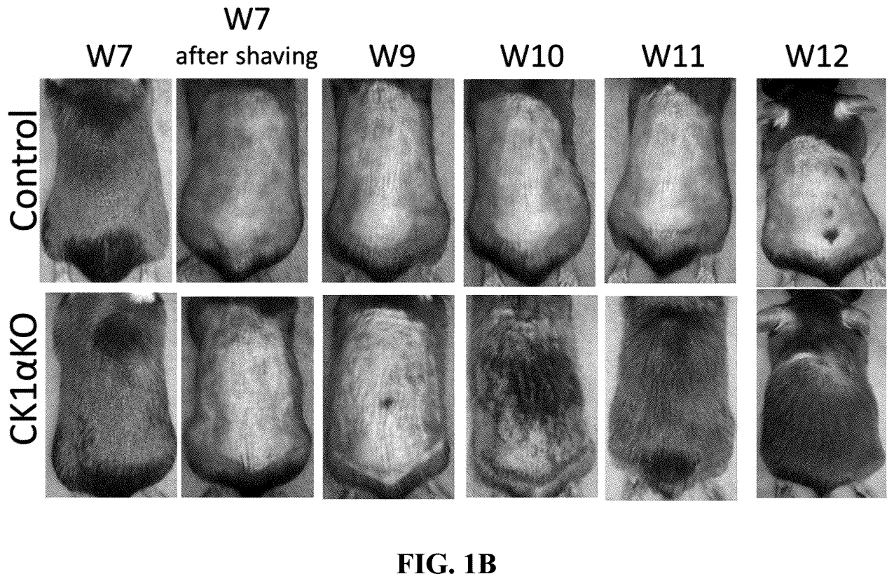

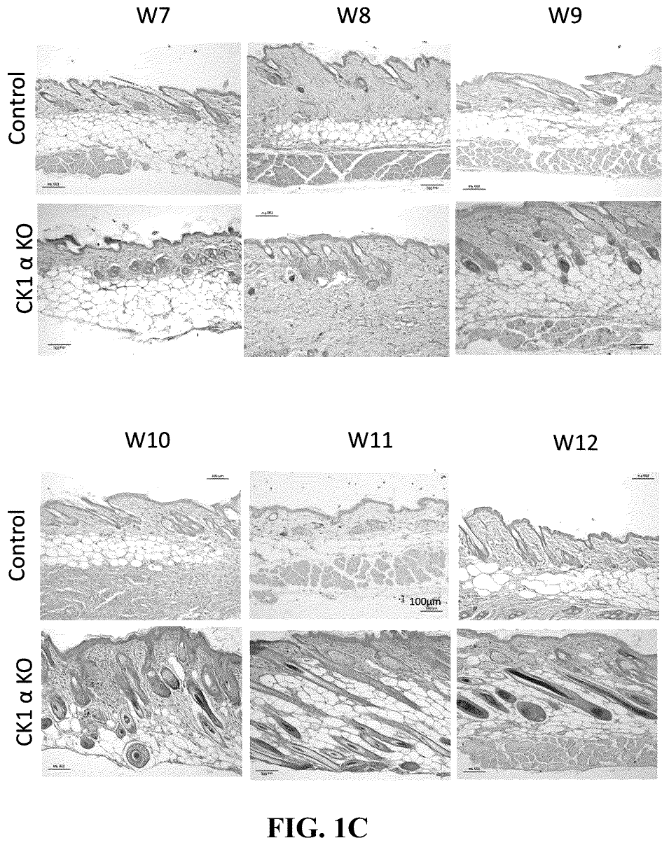

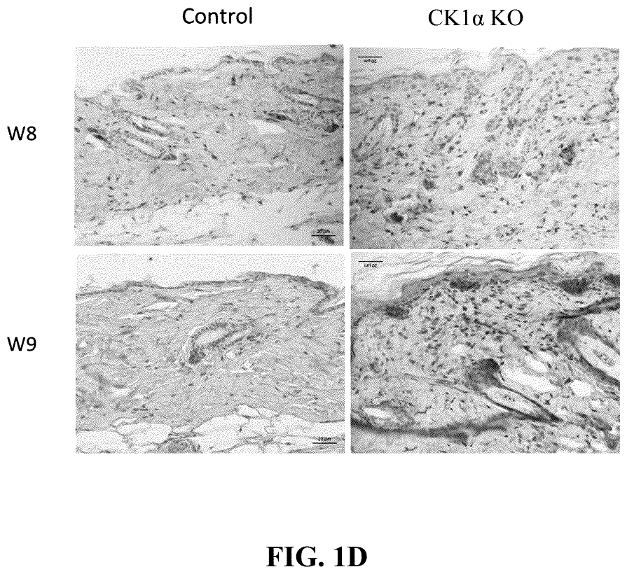

In the first study, K14-Cre-ERT2-CK1α^{fl/fl} a adult mice at 7 weeks old were used for intraperitoneal (i.p.) tamoxifen (TMX) induction to ablate CK1α expression in keratinocytes. FIG. 1A shows the study design, with the back hair of mice shaved at week 7 and TMX i.p. injection at a daily dose of 100 mg/kg on day 1 and day 2 after shaving. The phenotype and histology of mice skin sections were recorded every week from week 7 to week 12, as shown in FIG. 1B and FIG. 1C, respectively. It was shown that in control mice (N=6), hair follicles of the mice back skin maintained in a telogen phase from week 7 to week 12. However, in CK1α-ablated mice (CK1αKO, N=6), an early anagen phase of hair follicles appeared as early as in week 8 or 9 and continuously developed into full anagen during week 10 to week 12. Wnt/β-catenin pathway operates on hair follicle precursor cells and serves as a proximal signal for the telogen-anagen transition. As shown in FIG. 1D, increased β-catenin staining was demonstrated in CK1α-ablated mice skins in week 8 and week 9, compared to that in control mice.

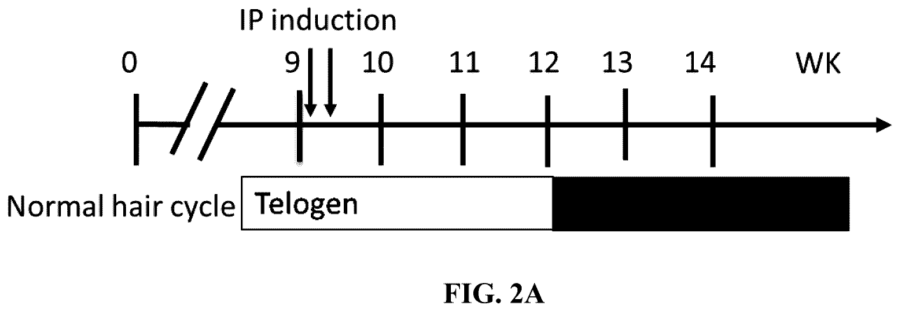

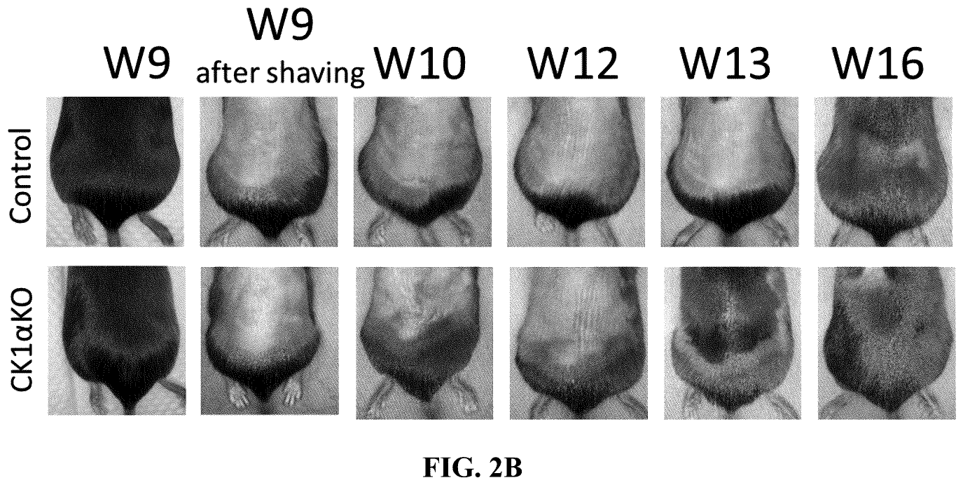

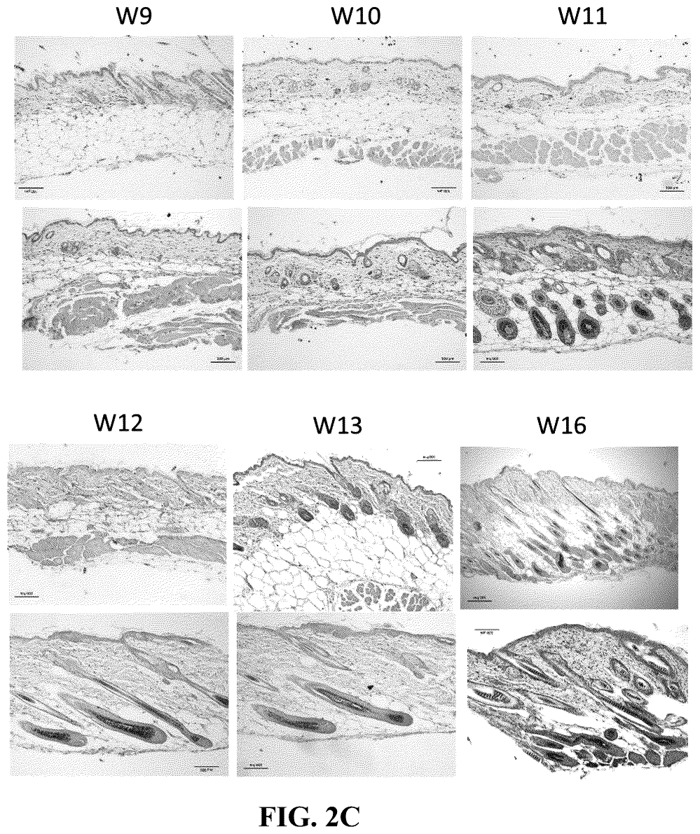

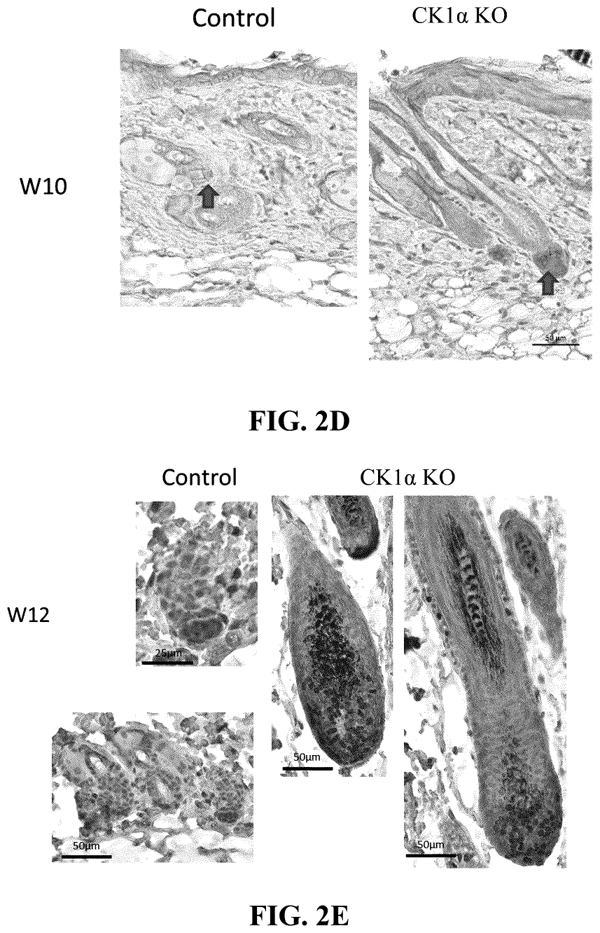

In the second study, K14-Cre-ERT2-CK1α^{fl/fl} adult mice at 9 weeks old were used for intraperitoneal (i.p.) tamoxifen (TMX) induction to ablate CK1α expression in keratinocytes. FIG. 2A shows the study design, with the back hair of mice shaved at week 9 and TMX i.p. injection at a daily dose of 100 mg/kg on day 1 and day 2 after shaving. The phenotypes of the mice were recorded from week 9 to week 16, as shown in FIG. 2B, respectively. It was shown that in control mice (N=3), hair follicles of the mice back skin maintained in a telogen phase from week 9 to week 12, with an anagen phase starting in week 13. In week 16, visible hair was observed on the back of the control mice. By contrast, in CK1α-ablated mice (CK1αKO, N=3), an early anagen phase of hair follicles appeared between week 10 and week 11, with visible hairs grown starting in week 13. In week 16, the CK1α-ablated mice backs were fully covered with hair. FIG. 2C shows the histology of mice skin sections with H&E staining. It was shown that before i.p. TMX induction in week 9, both the normal mice and CK1αKO mice showed the hair follicles in a telogen phase. Then, a telogen phase continued from week 9 to week 11 or 12 in control mice with the anagen phase began at week 13 and fully developed at week 16. On the other hand, in CK1αKO mice, an anagen phase began at week 10 with continuously growing of hairs during week 11 to week 16. That is, CK1α inhibition during a telogen phase in keratinocytes induces and initiates an anagen phase of a hair cycle to start early. FIG. 2D shows the result of immunohistochemistry staining of β-catenin. Similar to the first study, increased β-catenin staining was demonstrated in CK1α-ablated mice skins in week 10, compared to that in control mice. FIG. 2E shows the result of immunohistochemical staining of 5-bromo-2′-deoxyuridine (BrdU), which labels the proliferative cells, where increased staining in the matrix of hair follicles was shown in CK1αKO mice compared to control mice.

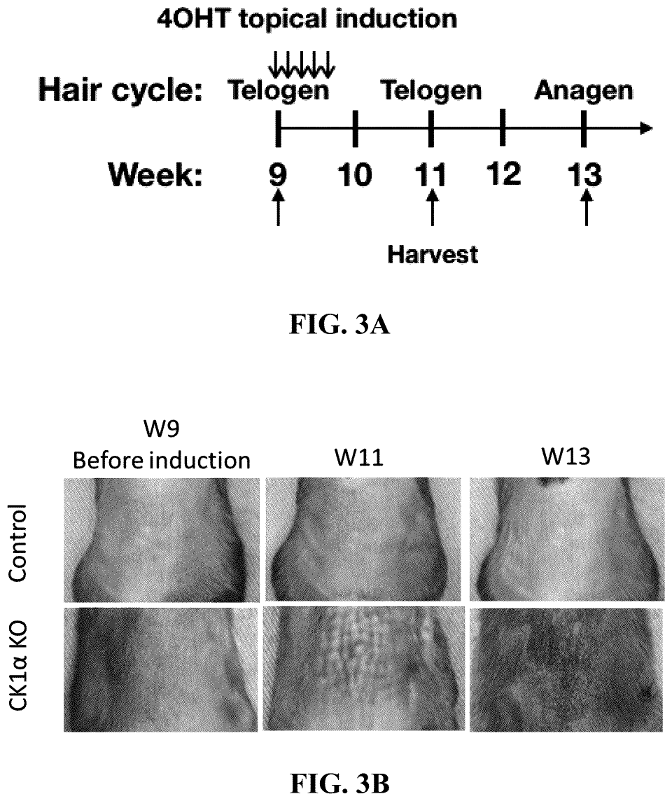

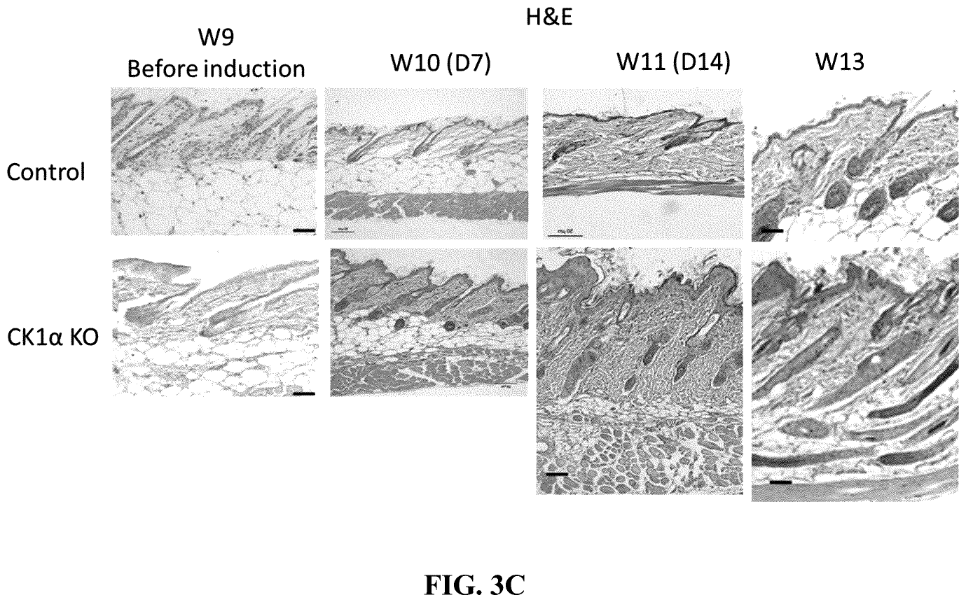

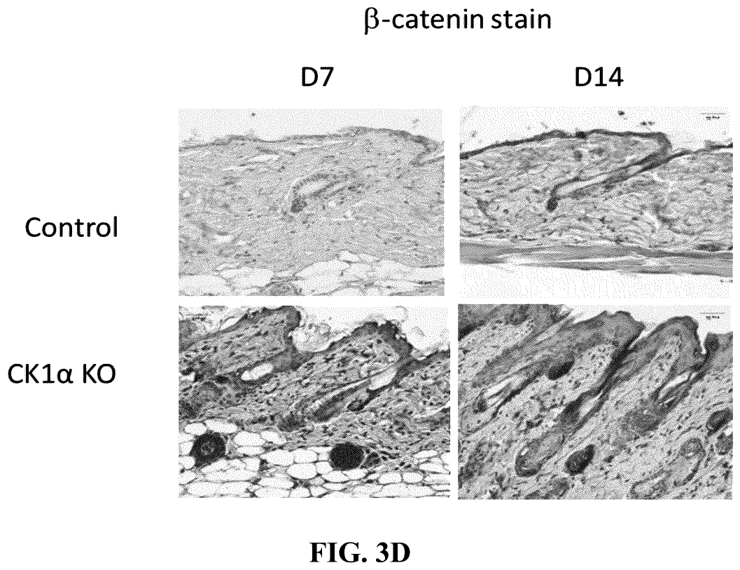

Next, 4-OH-TMX was used for topical induction of CK1α ablation in keratinocytes of the back skin of K14-Cre-ERT2-CK1α^{fl/fl} mice. As shown in FIG. 3A, mice at 9 weeks old were used with their back hair shaved at week 9, followed by 4-OH-TMX topical induction on the shaved back skin. FIG. 3B and FIG. 3C show the phenotypes and histology recorded from week 9 to week 13. In normal mice (N=5), a telogen phase was observed from week 9 to week 11, and anagen was noted to start in week 13. In CK1α-ablated mice (N=11), an anagen phase with elevated papules on skin surface in histology was noted on week 11, and visible hairs with fully developed anagen were noted in week 13. FIG. 3D shows the result of immunohistochemical staining of β-catenin. β-catenin expression in the hair germ at week 10 (W10) and week 11 (W11), which were day 7 (D7) and day 14 (D14) after induction of CK1α ablation, was shown to increase in CK1α KO mice compared to control mice. β-catenin nuclear staining was shown on the basal layer of epidermis, secondary hair germ and matrix, indicating activation of Wnt/β-catenin signal during the telogen-anagen transition.

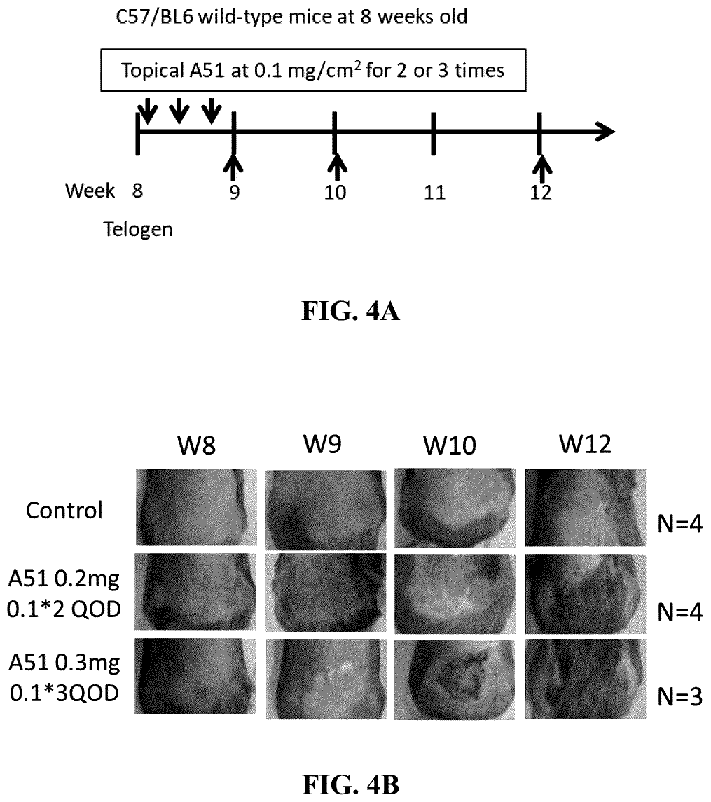

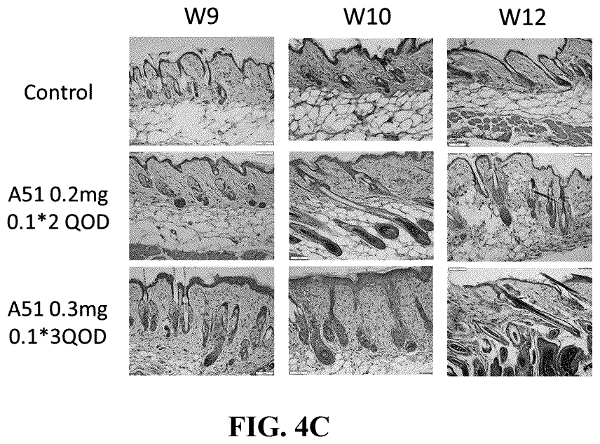

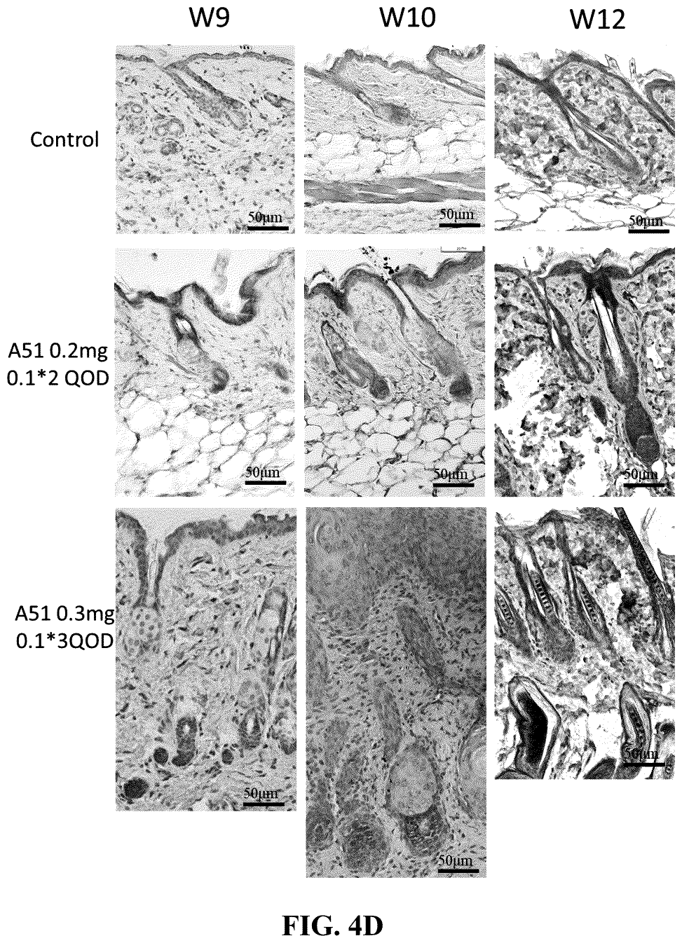

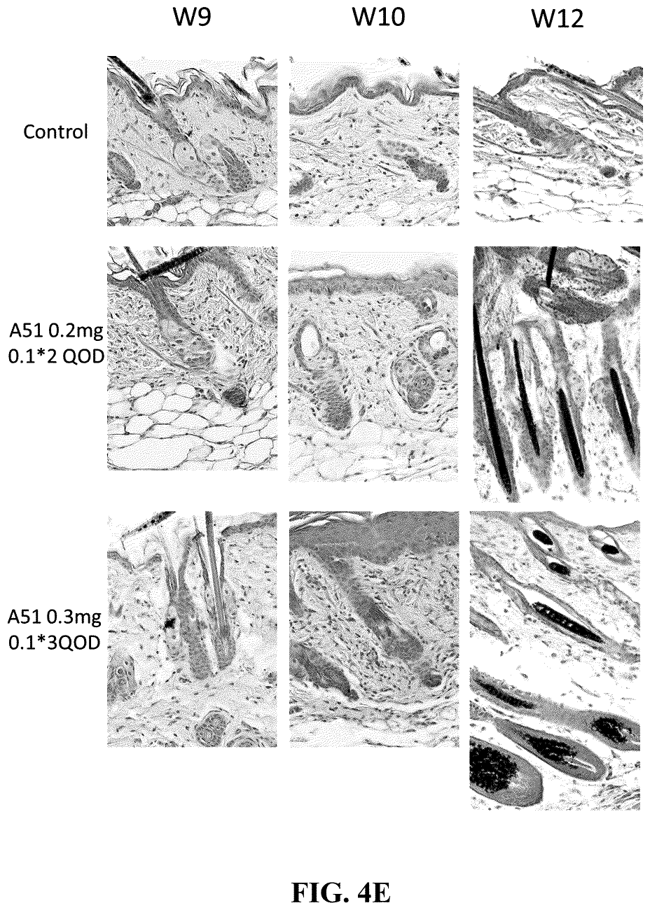

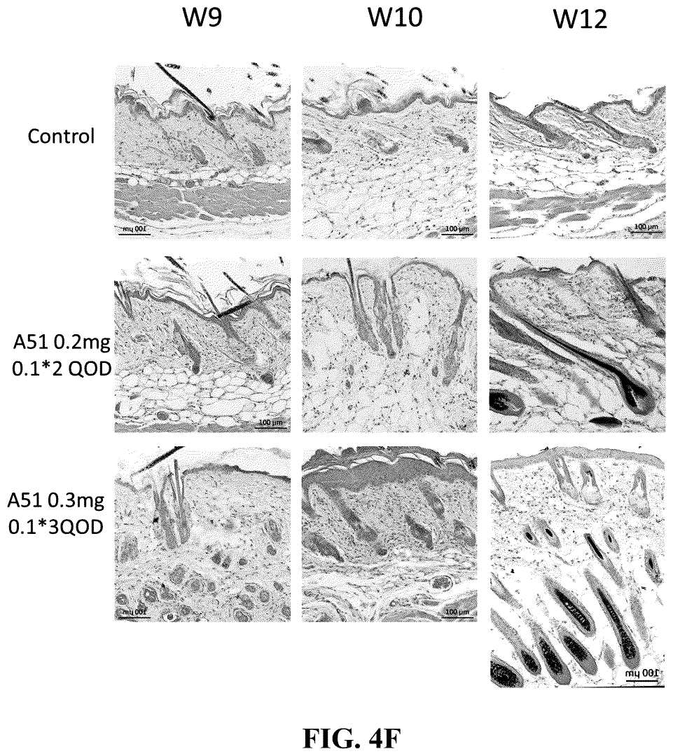

In addition, topical application of CK1 inhibitors on wild-type mice were carried out to inhibit CK1 expression in mice back skin. As shown in FIG. 4A, topical CK1 inhibitor, e.g., A51, at a concentration of 0.1 mg/cm² were given 2 or 3 times on the shaved back skin of mice at 8 weeks old. FIG. 4B shows starting of hair growth on the backs of mice topically treated with CK1 inhibitor A51 as early as in week 9 with most of the mice back covered with hair in week 12. FIG. 4C shows the H&E staining of back skins, and it was found that in the control group, hair follicles remained in a telogen phase during weeks 9, 10, and 12. However, an induced anagen phase was observed at week 9 in mice topically treated with 0.2 mg CKI at week 8, and mice topically treated with 0.3 mg CKI were induced with more advanced stage of anagen at week 9, with heavy pigmented hair shaft observed at week 12. That is to say, topical application of CK1 inhibitor is also able to induce and initiate an anagen phase of hair follicles from a telogen phase. FIG. 4D shows the result of immunohistochemical staining of β-catenin. Topical application of the CK1 inhibitor increased the expression of β-catenin in the hair germ, outer root sheath and epidermis, as compared to control mice. FIG. 4E shows the result of Fontana-Masson staining, which shows hair follicle pigmentation in CK1 inhibitor treated skin in addition to hair growth. Therefore, topical application of the CK1 inhibitor induces hair follicle growth and pigmented hair formation.

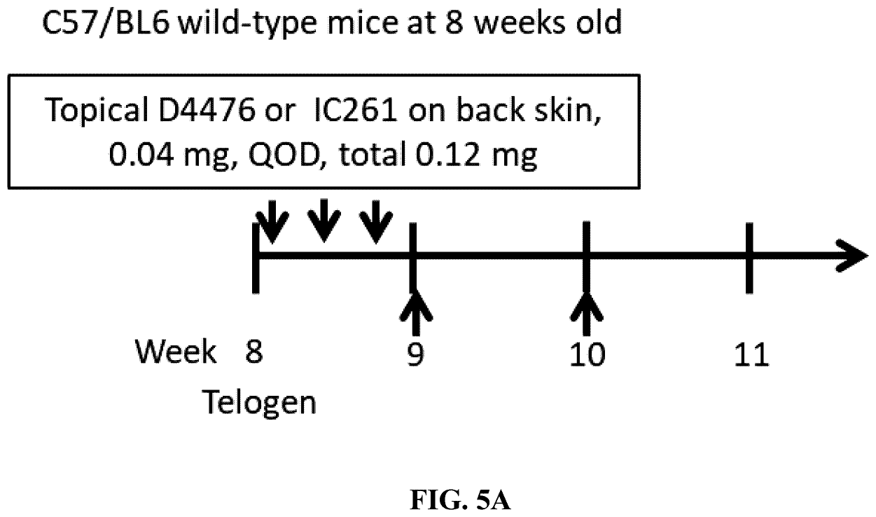

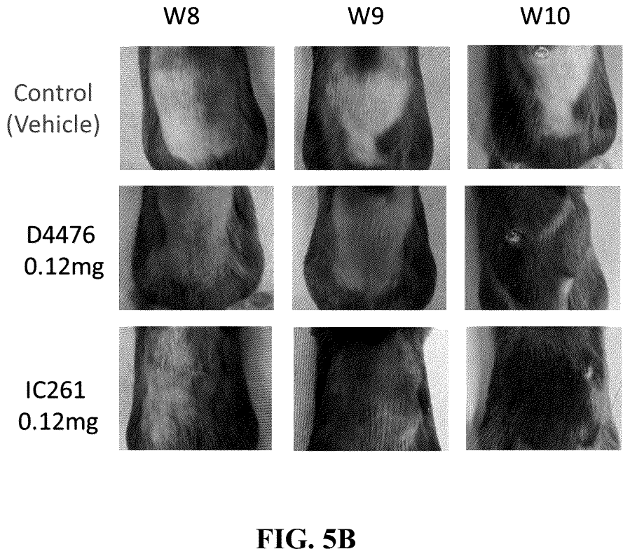

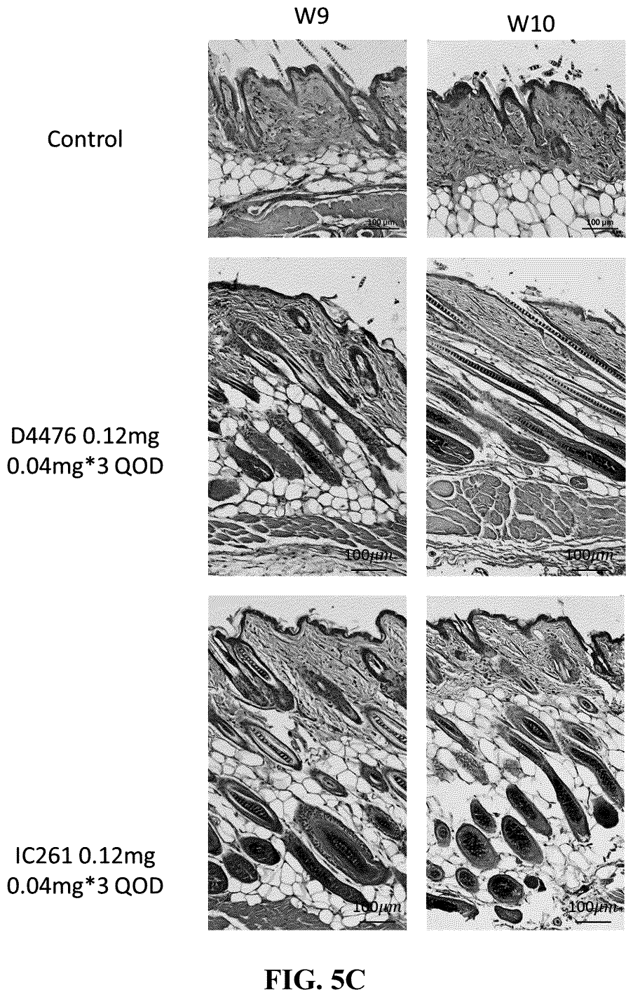

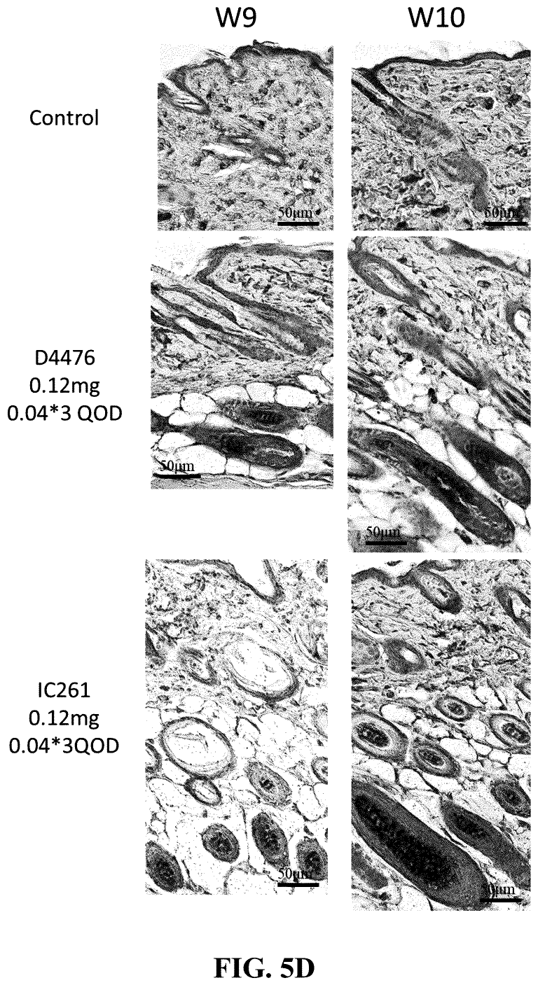

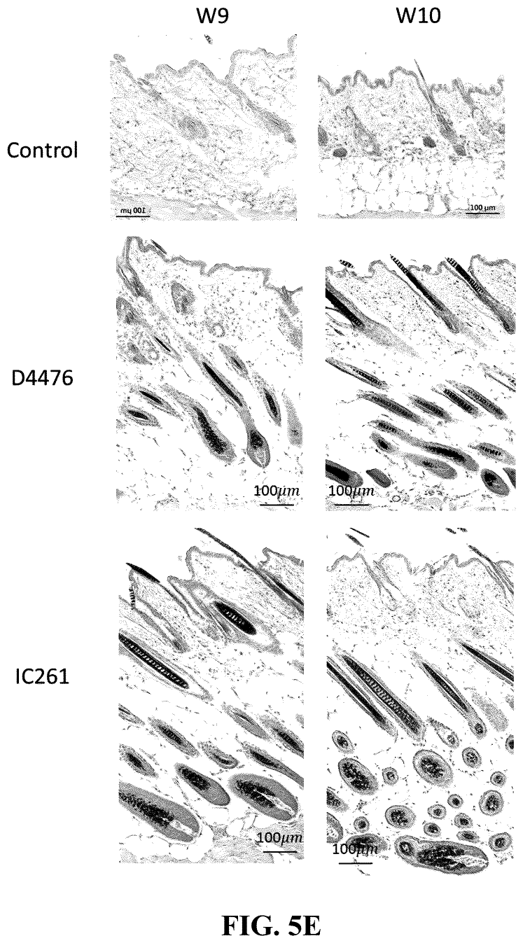

Other CK1 inhibitors including D4476 and IC261 were also tested. As shown in FIG. 5A, the CK1 inhibitors, D4476 and IC261, were topically applied to the shaved back skin of mice at 8 weeks old every other day, three times per week, with a total dose of 0.12 mg per 1 cm² of the back skin. FIG. 5B shows that topical application of D4476 or IC261 significantly induced hair growth on the back skin of the treated mice (N=3), which is a result of a hair cycle moving into anagen from telogen. FIG. 5C shows the H&E staining of back skins and it was found that topical application of a CK1 inhibitor induces an anagen phase of the hair cycle and observed formation of hair follicles showing characteristic of an anagen phase. FIG. 5D shows the result of immunohistochemical staining of β-catenin. Topical application of CK1 inhibitors, D4476 and IC261, also increased the expression of β-catenin in the matrix of hair follicle. FIG. 5E shows the result of Fontana-Masson staining with topical application of additional CK1 inhibitors such as D4476 and IC261 that induce hair follicle growth and pigmented hair formation.

### Example 2: Inhibition of CK1α Prolongs an Anagen Phase in a Hair Cycle

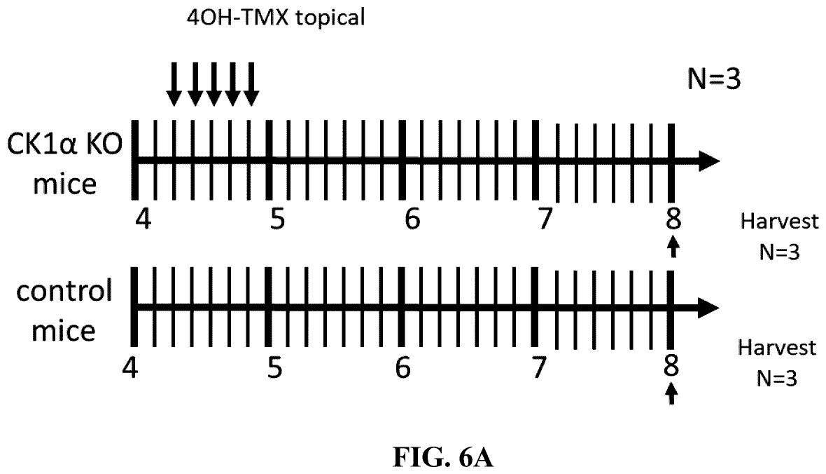

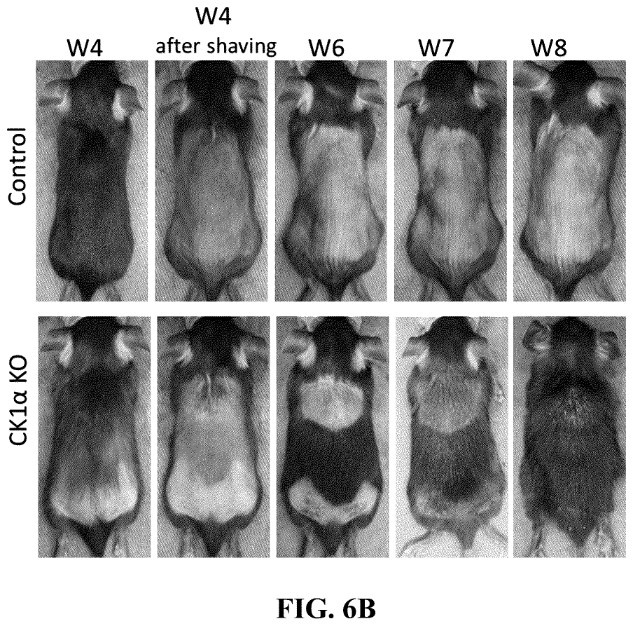

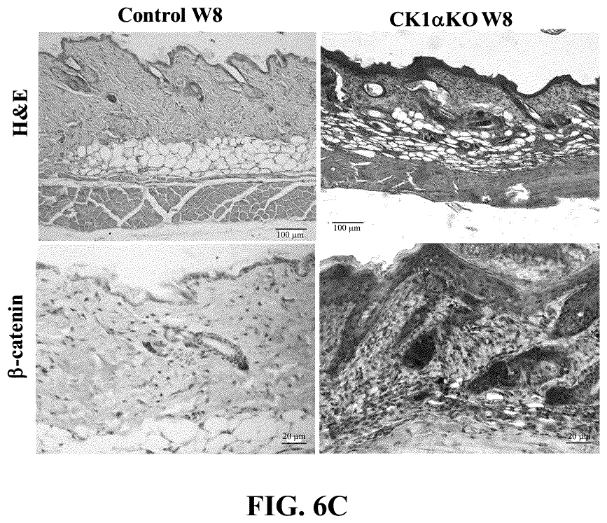

CK1α ablation carried out during an anagen phase of keratinocytes was found to increase the duration of an anagen phase in a hair cycle. 4-OH-TMX was used for topical induction of CK1α ablation in keratinocytes of the back skins of 4-weeks-old K14-Cre-ERT2-CK1α^{fl/fl} mice, as shown in FIG. 6A. Phenotypes were recorded from week 4 to week 8. As shown in FIG. 6B, it was found that in control mice (N=6), an anagen phase lasted from week 4 to week 5, followed by week 6 in a catagen phase, and stayed in a telogen phase from week 7 to week 8. In CK1α ablation mice (N=6), hairs appeared and started to cover the shaved back skins in weeks 6 to 8, indicating the anagen phase was prolonged. FIG. 6C shows the H&E staining and β-catenin staining of mice at week 8. It was found that control mice were in a telogen phase at week 8, but CK1α-ablated mice were still in an anagen phase at week 8 with enhanced β-catenin staining in the matrix of hair follicles.

### Example 3: Inhibition of CK1 Increases Hair Pigmentation

Inhibition of CK1 in keratinocytes was also found to increase pigmentation of hair. CK1 inhibitors including A51, D4476 and IC261 were topically applied to shaved back skin of mice at week 8, when the hair cycle was during the telogen phase. Then, Masson-Fontana staining was carried for assessing pigmentation of hair. Heavily pigmented hair follicles and shafts were found in hairs of the mice topically treated with A51, as shown in FIG. 4E, and also in hairs of the mice topically treated with D4476 and IC261, as shown in FIG. 5E.

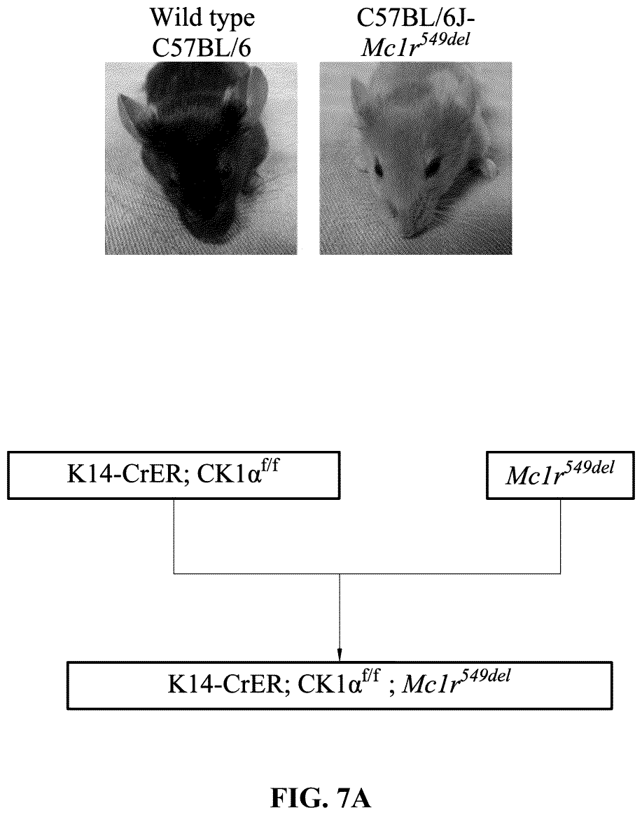

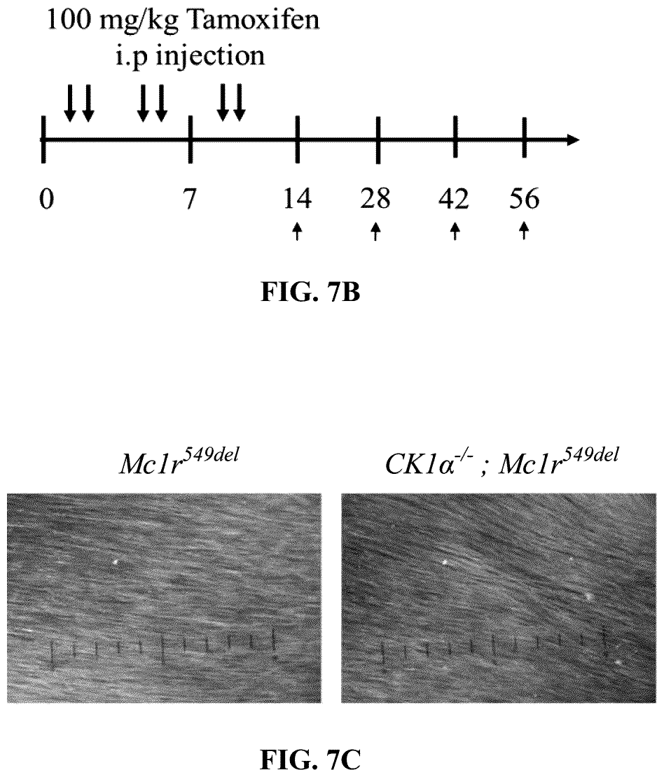

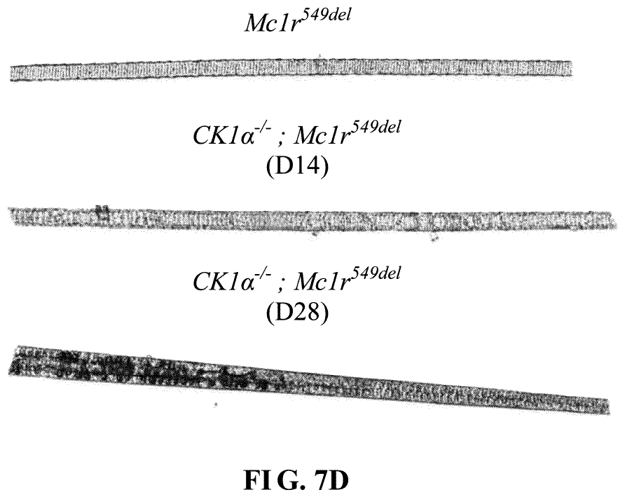

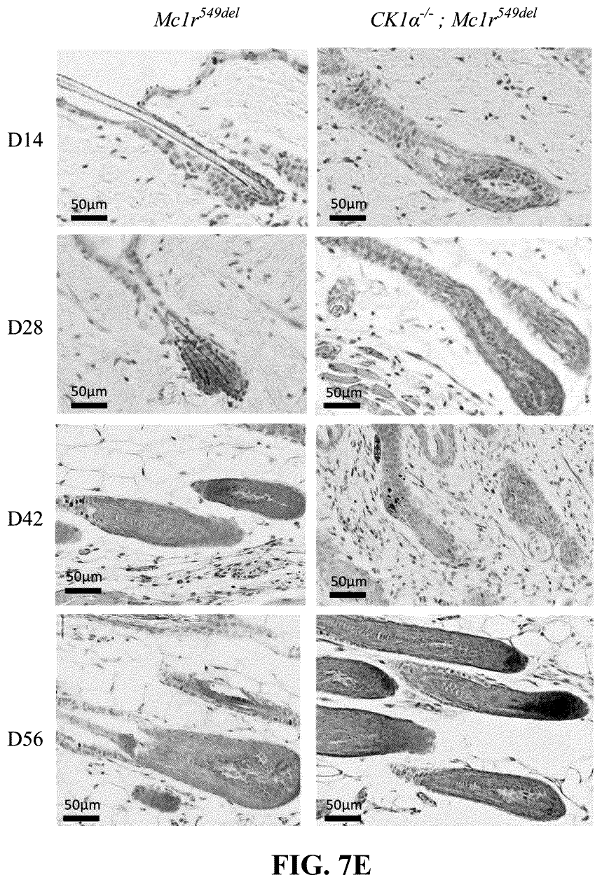

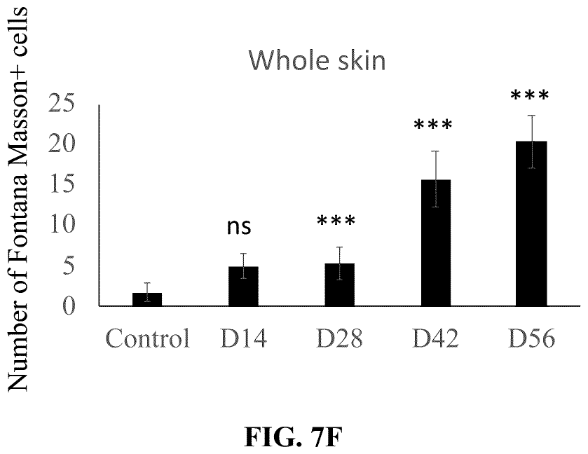

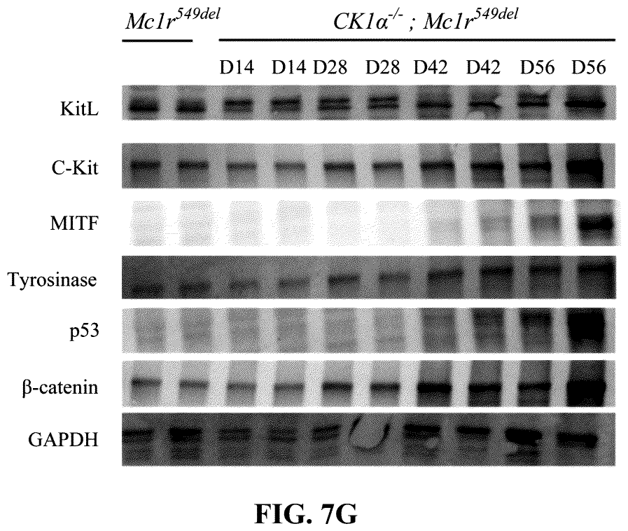

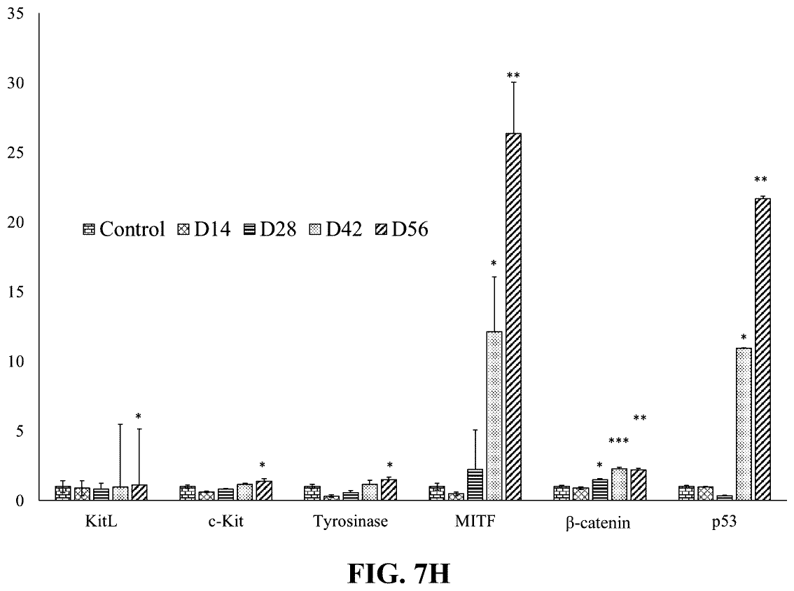

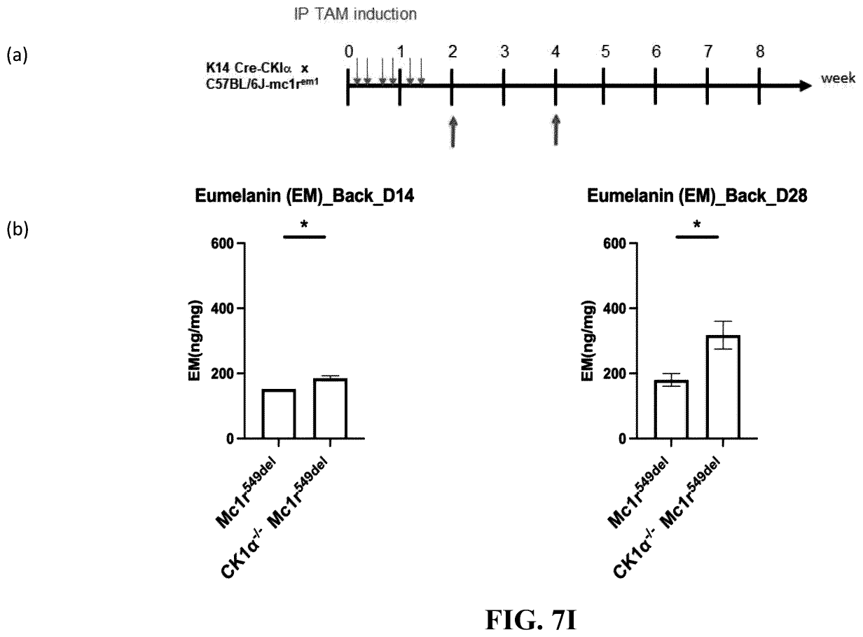

In addition to wild-type mice (black C57BL/6), Mc1r^{549del} mice were also used as an animal model to show increase of hair pigmentation by inhibition of CK1. The Mc1r^{549del} mice having a deletion of a single nucleotide at position 549 of Mc1r that leads to 12 amino acids out-of-frame mutation have been reported by Mountjoy, Robbins et al. 1992. The C57BL/6J-Mc1r^{549del} mice carrying a deletion of a single nucleotide at position 549 in the Mc1r gene were generated previously by a CRISPR/Cas9 system. The deletion causes a frame-shift mutation and leads to loss of function of the Mc1r protein, resulting in a yellow coat color in mice, due to exclusive synthesis of pheomelanin and failure to synthesize eumelanin by the melanocytes, as shown in the photograph of FIG. 7A. The Mc1r^{549del} mouse was then crossed with K14-CreER-CK1α^{f/f} mouse to generate K14-CreER-CK1α^{f/f};Mc1r^{549del} mouse, which can be induced to ablate CK1α in keratinocytes. As shown in FIG. 7B, 7-weeks-old mice were subjected to induction with intraperitoneal injection of tamoxifen at 100 mg/kg for a total of 6 times at days 1, 2, 5, 6, 8 and 9. Skin samples were harvested at days 14, 28, 42 and 56 for analysis. As shown in FIG. 7C, increased pigmentation of hairs was noted on Mc1r^{549del} mice with CK1α ablation in keratinocytes on day 28, compared to Mc1r^{549del} mice. The dissection microscopy results shown in FIG. 7D present a closer and clearer view on increased hair shaft pigmentation of Mc1r^{549del} mice with CK1α ablation in keratinocytes, on days 14 and 28, in comparison to Mc1r^{549del} mice. FIG. 7E shows the Fontana-Masson staining of the skin samples. It was shown that eumelanin intensity in the matrix and inner root sheath was increased, demonstrated by a time course analysis on Mc1r^{549del} mice with CK1α ablation in keratinocytes, which is not observed in Mc1r^{549del} mice. FIG. 7F shows the quantitated number of cells stained by Fontana-Masson staining in FIG. 7E. FIG. 7G and FIG. 7H show the Western blotting result and the corresponding quantitated expression levels, revealing stabilization of β-catenin and p53, and upregulation of KitL, c-Kit, MITF and tyrosinase. The KitL/C-Kit pathway was upregulated for the pigmentation of hair. FIG. 7I shows the eumelanin analysis on back skin of Mc1r^{549del} mouse versus CK1α^{-/+};Mc1r^{549del} mouse on day 14 and day 28, wherein panel a indicated the experimental design of eumelanin analysis. Intraperitoneal injection of tamoxifen to ablate CK1α on Mc1r^{549del} mice. Back skin of control versus CK1α knockout was harvest on day 14 and day 28 after induction. As shown in panel b, data showed increased eumelanin production in Mc1r^{549del} mice after CK1α knockout, since eumelanin on mouse back skin is mainly in the hair follicles, indicating CK1α inhibition on hairy skin can increase hair pigment.

### Example 4: Effect of CK1 Inhibition on Sebaceous Gland and Sebocytes

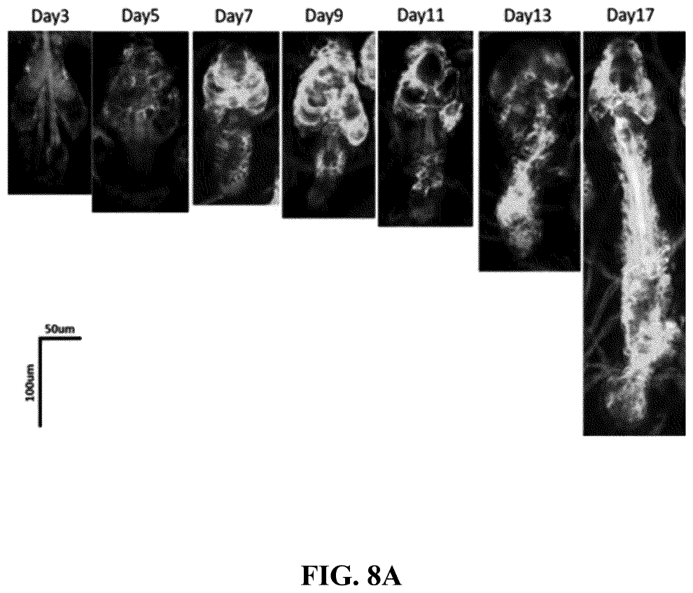

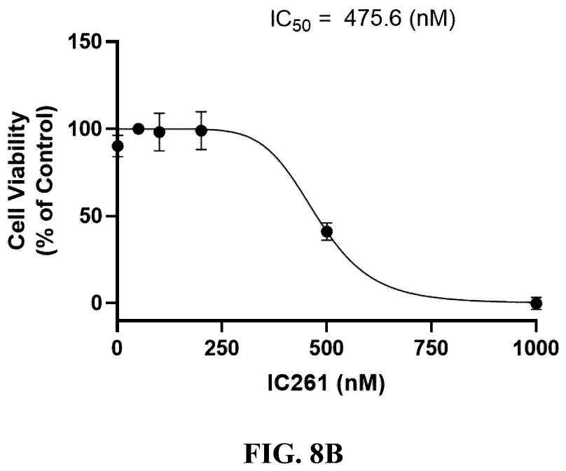

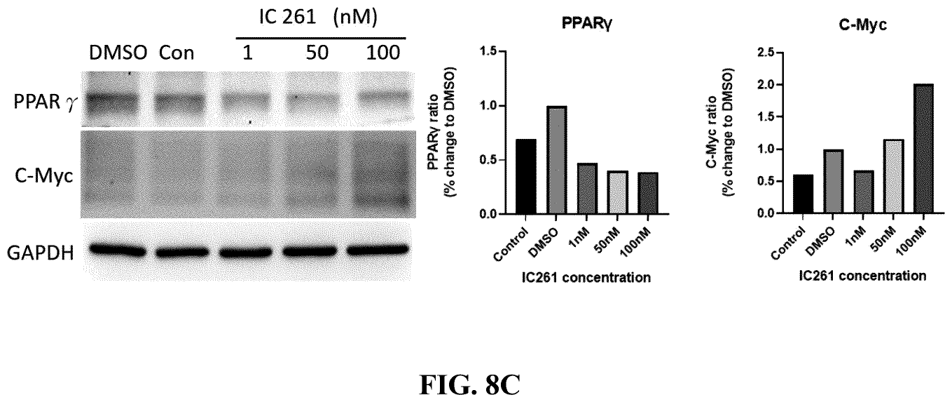

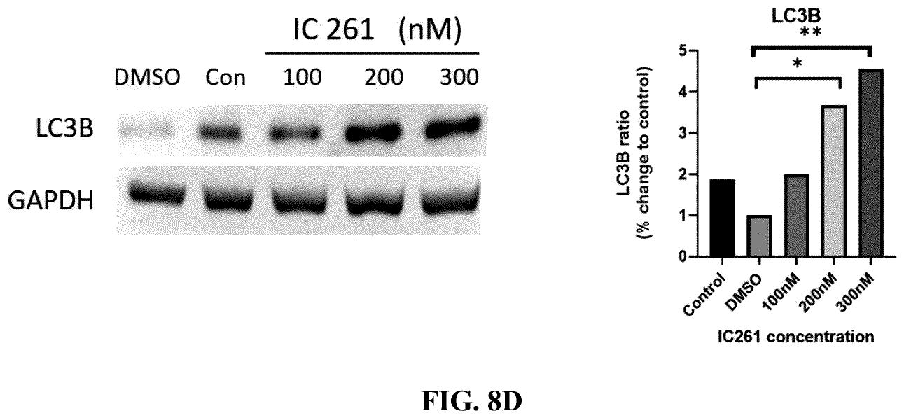

As shown in FIG. 8A, the intravital multiphotone imaging on the mouse ear skin of K14-CreER-CK1α; ROSAmT/mG mouse. Before induction, the keratinocytes of sebaceous gland (SG) and hair follicle (HF) express red fluorescence; after induction, the CK1α-ablated keratinocytes express green fluorescence. On day 3, the K14⁺ basal layer of sebaceous gland expresses green fluorescence, which can proliferate and differentiate into SG with green fluorescence unto day 17 during induced HF elongation. During CK1α inhibition on SG and HF, the size of SG was reduced, whereas the growth of HF was enhanced, indicating CK1α inhibition modulates the size of SG which may involve the fate specification of the multipotent stem cells in the pilosebaceous unit which includes follicle, hair shaft and sebaceous gland. As shown in FIG. 8B, SZ95 (human sebocyte cell line) was further treated with IC261 for 24 hr, wherein the cell number is 1.5×10⁴ cells/well, and IC50 is about 475.6 nM. As shown FIG. 8C, SZ95 cell culture presented sebocytes with various differentiation stages; PPARγ is a representative biomarker for adipogenesis in sebocytes, while c-Myc is a biomarker for undifferentiated sebocytes. Taken together, IC261 treatment induces PPARγ decreased and c-Myc increased, indicating its effect on inhibiting adipogenesis or differentiation on sebocytes. As shown in FIG. 8D, LC3B is a marker for autophagy. Overall, IC261 treatment on SZ95 cells induces LC3B increase with a dose dependent manner, indicating IC261 induces autophagy in sebocytes.

### Example 5: Beta-Catenin Favors Differentiation Toward Hair Follicle Stem Cells Over Sebaceous Gland Stem Cells

As illustrated in FIG. 1D and FIG. 2D, elevated β-catenin staining was observed in the skin of CK1α-ablated mice compared to that in control mice, indicating increased beta-catenin expression in keratinocytes in the presence of CKI. Additionally, pluripotent stem cells located beneath the entrance of the sebaceous gland duct within the hair follicle exhibit a trend to undergo differentiation towards hair follicle stem cells when exposed to increased beta-catenin levels. Consequently, under the influence of increased beta-catenin, pluripotent stem cells preferentially differentiate into the lineage of hair follicle stem cells rather than sebaceous gland stem cells. These results suggest that CKI may govern the determination of pluripotent stem cell fate, as evidenced by the differentiation tendencies observed in hair follicle stem cells and sebaceous gland stem cells.

Those skilled in the art will readily observe that numerous modifications and alterations of the present disclosure may be made while retaining the teachings of the invention described herein. Accordingly, the embodiments described are intended to cover the modifications and alterations within the scope of the present disclosure, rather than to limit the present disclosure. The scope of the claims therefore should be accorded the broadest interpretation so as to encompass all such modifications and alterations.

What is claimed is:

1. A method for regulating sebaceous gland on an area of skin in a subject in need thereof, comprising topically administering to the subject a casein kinase 1 inhibitor.

2. The method of claim 1, wherein the casein kinase 1 is selected from the group consisting of D4476, IC261, CK17, a compound represented by following general formula I, a compound of following formula II, a compound of following formula III, a compound of following formula IV, a compound of following formula V, and a compound of following formula VI: 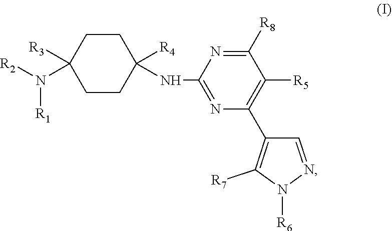
wherein:
R₁ and R₂ are each independently selected from the group consisting of H, straight or branched C1-C8 alkyl, straight or branched C1-C5 alkoxy, straight or branched C1-C5 acyl, C5-C15 aryl, and C3-C7 heteroaryl each optionally substituted by at least one of halide, hydroxyl, ester, ether, C5-C15 aryl, C3-C7 heteroaryl, and amide; or R₁ and R₂ together with the nitrogen atom they are connected to form a 4 to 7 membered saturated, unsaturated or aromatic ring optionally including at least one of N, O, NH, C═N, C═O and SO₂ and optionally substituted with at least one of straight or branched C1-C5 alkyl, C5-C15 aryl, C3-C7 heteroaryl, hydroxyl, halide and cyano;
R₃ and R₄ are each independently selected from the group consisting of H, straight or branched C1-C8 alkyl optionally substituted by at least one of halide, hydroxyl, alkoxy, C5-C15 aryl, C3-C7 heteroaryl, ester and amide; or
R₁ or R₂ together with R₃ and the carbon and nitrogen atom they are each connected to form a 4 to 7 membered saturated, unsaturated or aromatic ring optionally including at least one of N, NH, O, C═N, C═O, and SO₂, and optionally substituted with at least one of straight or branched C1-C5 alkyl, C5-C15 aryl, C3-C7 heteroaryl, hydroxyl, carbonyl, and halide;
R₅ and R₈ are each independently selected from the group consisting of H, halide, straight or branched C1-C8 alkyl, straight or branched C2-C8 alkenyl, and straight or branched C2-C8 alkynyl optionally substituted by at least one halide;
R₆ is selected from straight or branched C1-C8 alkyl, straight or branched C2-C8 alkenyl, straight or branched C2-C8 alkynyl, C5-C10 cycloalkyl, and saturated or unsaturated 4 to 6 membered heterocycle optionally substituted by at least one of straight or branched C1-C8 alkyl, C3-C7 cycloalkyl, 4 to 6 membered heterocycle, C5-C15 aryl, C3-C7 heteroaryl, halide, hydroxyl, and C1-C5 alkyl halide;
R₇ is selected from the group consisting of straight or branched C1-C8 alkyl, straight or branched C2-C8 alkenyl, and straight or branched C2-C8 alkynyl optionally substituted by at least one of C3-C7 cycloalkyl, 4 to 6 membered heterocycle, C5-C15 aryl, C3-C7 heteroaryl, halide, hydroxyl, and C1-C5 alkyl halide;

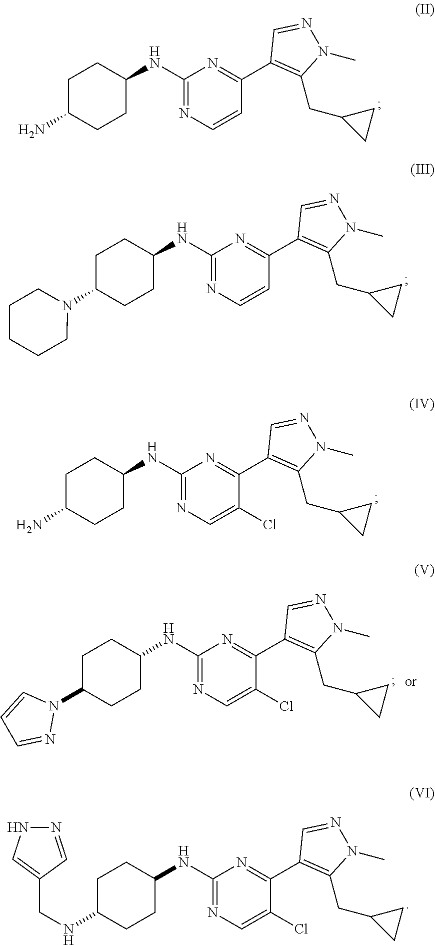

3. The method of claim 1, wherein the subject suffers from a sebaceous gland-related disorder.

4. The method of claim 3, wherein the sebaceous gland-related disorder is a sebaceous gland dysfunction and/or a sebum overproduction-related disorder.

5. The method of claim 4, wherein the sebum overproduction-related disorder is induced by drug, infection, or hormones.

6. The method of claim 4, wherein the sebum overproduction-related disorder is selected from the group consisting of an acne, a seborrhea, a dermatitis, and an oily skin.

7. The method of claim 6, wherein the acne is a hormone-induced acne.

8. The method of claim 6, wherein the dermatitis is an infection-induced dermatitis or a seborrheic dermatitis.

9. The method of claim 1, wherein the subject suffers from a hair loss.

10. The method of claim 9, wherein the hair loss is a hair loss caused by nutritional deficiency, a drug-induced hair loss, a radiation-induced hair loss, a stress-induced hair loss, a genetic hair loss, an aging hair loss, or a disease-induced hair loss.

11. The method of claim 10, wherein the drug-induced hair loss is induced by a chemotherapy drug, lithium, arsenic, bismuth, boric acid, thallium, colchicine, retinoid, heparin, warfarin, β-blocker, an angiotensin-converting enzyme (ACE) inhibitor, a hormone, valproic acid, carbamazepine, phenytoin, cimetidine, an antithyroid drug, a cholesterol-lowering drug, interferon, an anti-infective agent, amphetamine, an antidepressant, an anti-fungal agent, an anti-seizure agent, a birth control agent, a vitamin A-based medication, a medication for Parkinson's disease, a medication for stomach or a nonsteroidal anti-inflammatory drug.

12. The method of claim 11, wherein the disease-induced hair loss is caused by an autoimmune disease, a thyroid disorder, a metabolic syndrome, an infection, or a cancer.

13. The method of claim 12, wherein the autoimmune disease is alopecia areata, lupus erythematosus, Sicca syndrome, scleroderma, Crohn's disease, inflammatory bowel disease, or psoriasis.

14. The method of claim 9, wherein the subject suffers from alopecia.

15. The method of claim 14, wherein the alopecia is selected from the group consisting of androgenetic alopecia, alopecia areata, anagen effluvium, self-induced hair loss, telogen effluvium, and scarring alopecia.

16. The method of claim 1, wherein the casein kinase 1 is casein kinase 1a.
